(19) United States
(12) Patent Application Publication
(10) Pub. No.: US 2023/0263798 A1
(43) Pub. Date: Aug. 24, 2023

(54) COMPOSITIONS AND METHODS FOR TREATING THERAPY RESISTANT CANCER

(71) Applicant: The General Hospital Corporation, Boston, MA (US)

(72) Inventors: Shobha VASUDEVAN, Boston, MA (US); Syed Irfan Ahmad BUKHARI, Boston, MA (US); Samuel Spencer TRUESDELL, Boston, MA (US)

(21) Appl. No.: 18/024,182

(22) PCT Filed: Sep. 1, 2021
(86) PCT No.: PCT/US2021/048753

§ 371 Date: Mar. 1, 2023

Related U.S. Application Data
(60) Provisional application No. 63/072,963, filed on Sep. 1, 2020.

(51) Int. Cl. A61K 31/496; A61P 35/00; A61K 31/704; A61K 31/7068; A61K 31/502; A61P 35/02; A61K 31/337; A61K 31/357; A61K 31/155; A61K 31/7076; A61K 31/4545; A61K 31/343; A61K 31/519; A61K 31/4178; A61K 31/47; A61K 35/15; A61K 35/17; A61K 31/7105

(52) U.S. Cl. CPC A61K 31/496; A61K 31/155; A61K 31/337; A61K 31/343; A61K 31/357; A61K 31/4178; A61K 31/4545; A61K 31/47; A61K 31/502; A61K 31/519; A61K 31/704; A61K 31/7068; A61K 31/7076; A61K 31/7105; A61K 35/15; A61K 35/17; A61P 35/00; A61P 35/02

(57) ABSTRACT

Described herein are compositions and methods for treating cancer in a subject. Using the compositions and methods of the disclosure, a subject may be administered (i) an inhibitor and/or an overrider and (ii) a chemotherapeutic.

### CROSS-REFERENCE TO RELATED APPLICATIONS

This application claims benefit of U.S. Provisional Application No. 63/072,963, filed on Sep. 1, 2020, the contents of which are incorporated herein by reference in their entirety.

### FIELD OF THE INVENTION

This invention relates to methods of treating cancer, as well as compositions that may be used in such methods.

### BACKGROUND OF THE INVENTION

Quiescent (G0) cells are resistant to chemotherapy that induces DNA damage due to altered gene expression in such cells. Downregulation of canonical translation in such cells enables alternate post-transcriptional mechanisms to express specific mRNAs that mediate chemosurvival. In such chemoresistant cells, mTOR/Akt activity is inhibited while other stress signals, including the integrated stress response (ISR), are activated. These two effects suppress both rate limiting steps of canonical cap dependent translation: mTOR inhibition blocks mRNA recruitment via the canonical cap complex and activation of the ISR activates eukaryotic initiation factor 2-α (elF2α) kinases (e.g., protein kinase R (PKR), PKR-like endoplasmic reticulum kinase (PERK), heme-regulated inhibitor (HRI), and/or general control non-depressible 2 (GCN2)) that phosphorylate elF2α of the canonical tRNA recruiter elF2 complex, leading to inhibition of canonical initiator tRNA recruitment. In addition, chemotherapy stress induces downstream kinases that modify RNA binding proteins that alter RNA levels and translation. These changes in gene expression inhibit proliferation that is driven by canonical translation and permit non-canonical translation of genes that enable chemosurvival. Despite such findings, there remains a need for improved cancer therapies to overcome chemosurvival.

### SUMMARY OF THE INVENTION

The present invention relates to compositions and methods for the treatment of cancer. In a first aspect, the invention features a combination that includes: (i) trazodone and (ii) a chemotherapeutic.

In a further aspect, the invention features a combination that includes: (i) an integrated stress response (ISR) overrider, an ISR inhibitor, an adenosine deaminases acting on ribonucleic acid (ADAR) inhibitor, a protein kinase C (PKC) inhibitor, a poly adenosine diphosphate-ribose polymerase (PARP) inhibitor, a methyltransferase-like 3 (METTL3) inhibitor, or a one-carbon metabolism inhibitor; and (ii) a chemotherapeutic.

In some embodiments of any of the above aspects, the chemotherapeutic is paclitaxel, gemcitabine, cytarabine, doxorubicin, or etoposide.

In some embodiments, the combination includes an ISR overrider. In some embodiments, the ISR overrider includes trazodone or integrated stress response inhibitor (ISRIB).

In some embodiments, the combination includes an ISR inhibitor. In some embodiments, the ISR inhibitor includes metformin or phenformin.

In some embodiments, the combination includes an ADAR inhibitor. In some embodiments, the ADAR inhibitor includes 8-azaadenosine.

In some embodiments, the combination includes a PKC inhibitor. In some embodiments, the PKC inhibitor includes enzastaurin.

In some embodiments, the combination includes a PARP inhibitor. In some embodiments, the PARP inhibitor includes talazoparib.

In some embodiments, the combination includes a METTL3 inhibitor. In some embodiments, the METTL3 inhibitor includes an interfering RNA molecule. In some embodiments, the interfering RNA molecule is a small interfering RNA (siRNA). In some embodiments, the siRNA includes a target sequence having the nucleic acid sequence of CGTCAGTATCTTGGGCAAGTT (SEQ ID NO: 1). In some embodiments, the siRNA includes a sense strand having the nucleic acid sequence of CGUCAGUAUCUUGGGCAAGUU (SEQ ID NO: 2). In some embodiments, the siRNA includes an antisense strand having the nucleic acid sequence of AACUUGCCCAAGAUACUGACG (SEQ ID NO: 3). In some embodiments, the interfering RNA molecule is a short hairpin RNA (shRNA). In some embodiments, the shRNA includes a target sequence having the nucleic acid sequence of GCTGCACTTCAGACGAATTAT (SEQ ID NO: 4). In some embodiments, the METTL3 inhibitor includes rocaglates.

In some embodiments, the combination includes a one-carbon metabolism inhibitor. In some embodiments, the one-carbon metabolism inhibitor includes methotrexate, serine hydroxymethyltranferase inhibitor 1 (SHIN-1), bisantrene, or brequinar.

In some embodiments, the combination includes immune cells. In some embodiments, the immune cells are monocytes (e.g., CD14+ monocytes), T cells (e.g., CD8+ T cells), or Natural Killer cells (e.g., NK92 cells).

In a further aspect, the invention features a method of treating cancer in a subject, the method including administering to the subject: (i) trazodone and (ii) a chemotherapeutic.

In a further aspect, the invention features a method of treating cancer in a subject, the method including administering to the subject: (i) an ISR overrider, an ISR inhibitor, an ADAR inhibitor, a PKC inhibitor, a PARP inhibitor, a METTL3 inhibitor, or a one-carbon metabolism inhibitor; and (ii) a chemotherapeutic.

In some embodiments, the cancer includes acute myeloid leukemia, liver cancer (e.g., hepatocellular carcinoma or hepatoblastoma), gastric cancer, lung cancer (e.g., non-small cell lung cancer), colorectal cancer, bladder cancer, pancreatic cancer, glioblastoma, prostate cancer, or breast cancer (e.g., triple negative breast cancer or hormone-positive breast cancer). In some embodiments, the cancer is acute myeloid leukemia. In some embodiments, the cancer is breast cancer (e.g., triple negative breast cancer or hormone-positive breast cancer).

In some embodiments, the chemotherapeutic is paclitaxel, gemcitabine, cytarabine, doxorubicin, or etoposide.

In some embodiments, the method includes administering an ISR overrider. In some embodiments, the ISR overrider includes trazodone or ISRIB.

In some embodiments, the method includes administering an ISR inhibitor. In some embodiments, the ISR inhibitor includes metformin or phenformin.

In some embodiments, the method includes administering an ADAR inhibitor. In some embodiments, the ADAR inhibitor includes 8-azaadenosine.

In some embodiments, the method includes administering a PKC inhibitor. In some embodiments, the PKC inhibitor includes enzastaurin.

In some embodiments, the method includes administering a PARP inhibitor. In some embodiments, the PARP inhibitor includes talazoparib.

In some embodiments, the method includes administering a METTL3 inhibitor. In some embodiments, the METTL3 inhibitor includes an interfering RNA molecule. In some embodiments, the interfering RNA molecule is a siRNA. In some embodiments, the siRNA includes a target sequence having the nucleic acid sequence of SEQ ID NO: 1. In some embodiments, the siRNA includes a sense strand having the nucleic acid sequence of SEQ ID NO: 2. In some embodiments, the siRNA includes an antisense strand having the nucleic acid sequence of SEQ ID NO: 3. In some embodiments, the interfering RNA molecule is a shRNA. In some embodiments, the shRNA includes a target sequence having the nucleic acid sequence of SEQ ID NO: 4. In some embodiments, the METTL3 inhibitor includes rocaglates.

In some embodiments, the method includes administering a one-carbon metabolism inhibitor. In some embodiments, the one-carbon metabolism inhibitor includes methotrexate, SHIN-1, bisantrene, or brequinar.

In some embodiments, trazodone and the chemotherapeutic are co-administered. In some embodiments, trazodone is administered prior to the chemotherapeutic.

In some embodiments, the ISR inhibitor, ADAR inhibitor, PKC inhibitor, PARP inhibitor, METTL3 inhibitor, or one-carbon metabolism inhibitor and the chemotherapeutic are co-administered. In some embodiments, the ISR inhibitor, ADAR inhibitor, PKC inhibitor, PARP inhibitor, METTL3 inhibitor, or one-carbon metabolism inhibitor is administered prior to the chemotherapeutic.

In some embodiments, the method further includes the step of administering immune cells to the subject. In some embodiments, the immune cells are monocytes (e.g., CD14+ monocytes), T cells (e.g., CD8+ T cells), or Natural Killer cells (e.g., NK92 cells).

In another aspect, the invention features use of an inhibitor of METTL3 or an inhibitor of METTL14 or an inhibitor of elF2α phosphorylation to decrease cancer cell resistance to chemotherapy.

In another aspect, the invention features a method of decreasing cancer cell resistance to chemotherapy in a patient including administering an inhibitor of METTL3 or an inhibitor of METTL14 or an inhibitor of elF2α phosphorylation to the patient in an amount sufficient to reduce the resistance of the cancer cell to chemotherapy.

In another aspect, the invention features a method of treating a cancer in a patient including co-administrating (i) a chemotherapeutic agent and (2) a METTL3 or METTL14 inhibitor or a elF2α phosphorylation inhibitor to the patient.

In some embodiments, the inhibitor is selected from trazodone, ISRIB, enzastaurin, compounds disclosed in PCT Patent Publications WO2014/144952 and WO2014/161808 (each of which are incorporated by reference in their entirety), miR600 and other small interfering RNAs disclosed in or based upon the METTL3 sequence disclosed in Chinese Patent CN107349217 (which is incorporated by reference in its entirety), or combinations of these.

In another aspect, the invention features any and all compositions, articles of manufacture, methods and uses disclosed and/or described in this specification.

In another aspect, the invention features a combination that includes: (i) an ISR overrider, an ISR inhibitor, a PKC inhibitor, a METTL3 inhibitor, or a one-carbon metabolism inhibitor; (ii) an ADAR inhibitor or a PARP inhibitor; and (iii) a chemotherapeutic.

In yet another aspect, the invention features a method of treating cancer in a subject, the method including (i) an ISR overrider, an ISR inhibitor, a PKC inhibitor, a METTL3 inhibitor, or a one-carbon metabolism inhibitor; (ii) an ADAR inhibitor or a PARP inhibitor; and (iii) a chemotherapeutic.

According to the methods described herein, a chemotherapeutic and an inhibitor (and/or an overrider) may be co-administered to a subject. Such co-administration typically involves administering a chemotherapeutic and inhibitor (and/or an overrider) together. In some embodiments, co-administration involves administering first a chemotherapeutic followed by administering within, for example, 1 minute, 5 minutes, 10 minutes, 20 minutes, or 30 minutes an inhibitor (and/or an overrider). In other embodiments, co-administration involves administering first an inhibitor (and/or an overrider) followed by administering within, for example, 1 minute, 5 minutes, 10 minutes, 20 minutes, or 30 minutes a chemotherapeutic.

Still further, according to the methods described herein, a subject may be administered an inhibitor (and/or an overrider) prior to receiving a chemotherapeutic. In some embodiments, the subject is administered the inhibitor, for example, 45 minutes, 50 minutes, 55 minutes, 1 hour, 2 hours, 3 hours, 4 hours, 5 hours, 6 hours, 7 hours, 8 hours, or even 12 to 24 hours prior to receiving the chemotherapeutic. In other embodiments, the subject is administered the overrider, for example, 45 minutes, 50 minutes, 55 minutes, 1 hour, 2 hours, 3 hours, 4 hours, 5 hours, 6 hours, 7 hours, 8 hours, or even 12 to 24 hours prior to receiving the chemotherapeutic.

In other embodiments, an inhibitor (and/or an overrider) is not administered to a subject who has received a chemotherapeutic. For example, an inhibitor (and/or overrider) is not administered 45 minutes, 50 minutes, or 1 hour or more after the subject has been administered the chemotherapeutic.

In certain embodiments, chemotherapeutics are administered to a subject or a patient according to standard methods known in the art (e.g., orally (e.g., a pill or capsule) or intravenously).

In yet other embodiments, the inhibitors or overriders are administered to a subject or a patient according to standard methods known in the art (e.g., orally (e.g., a pill or capsule) or intravenously).

Other features and advantages of the invention will be apparent from the following description of the preferred embodiments thereof, and from the claims.

### DEFINITIONS

As used herein, the term “inhibitor” refers to an agent (e.g., a small molecule (e.g., metformin, phenformin, 8-azaadenosine, enzastaurin, or talazoparib)), peptide fragment, protein, antibody, antigen-binding fragment thereof, or a nucleic acid (e.g., an interfering RNA molecule, such as a small hairpin RNA or a small interfering RNA)) that binds to, and/or otherwise suppresses the activity of, a target molecule.

As used herein, the term “overrider” refers to an agent (e.g., a small molecule (e.g., trazodone or integrated stress response inhibitor (ISRIB)), peptide fragment, protein, antibody, antigen-binding fragment thereof, a nucleic acid (e.g., an interfering RNA molecule, such as a small hairpin RNA or a small interfering RNA)) that functions downstream of a target molecule. Accordingly, unlike an inhibitor that in general binds and suppresses activity of the target molecule, an overrider reactivates other functions downstream to suppress the impact of the upstream target.

As used herein, the term “adenosine deaminases acting on ribonucleic acid” or “ADAR” refers to an RNA editing enzyme that binds to double-stranded RNA and converts adenosine to inosine through deamination.

As used herein, the term “integrated stress response” or “ISR” refers to the common adaptive pathway that eukaryotic cells activate in response to stress stimuli. The ISR involves the phosphorylation of eukaryotic translation initiation factor 2 alpha (elF2α) by members of the elF2α kinase family: protein kinase R (PKR), PKR-like endoplasmic reticulum kinase (PERK), heme-regulated inhibitor (HRI), and/or general control non-depressible 2 (GCN2). Phosphorylation of elF2α leads to a decrease in global protein synthesis and the induction of selected genes that together promote cellular recovery, which can cause tumor survival.

As used herein, the term “methyltransferase-like 3” or “METTL3” refers to the RNA methyltransferase involved in the posttranscriptional methylation of internal adenosine residues in eukaryotic mRNAs and involved in mRNA biogenesis, decay, and translation control through N(6)-methyladenosine (m(6)A) modification.

As used herein, the term “one-carbon metabolism” refers to a series of interlinking metabolic pathways that include the methionine and folate cycles that are central to cellular function, providing one-carbon units (methyl groups) for the synthesis (and modification by methylation or SAM (S-adenosyl-L-methionine)) of DNA, polyamines, amino acids, creatine, and phospholipids.

As used herein, the term “poly adenosine diphosphate-ribose polymerase” or “PARP” refers to a family of enzymes that catalyze the transfer of adenosine diphosphate-ribose to target proteins and RNAS. PARPs play a role in DNA repair, chromatin modulation, mitosis, cell death, telomere length, and intracellular metabolism.

As used herein, the term “protein kinase C” or “PKC” refers to a family of serine/threonine kinases that regulate various cellular functions including proliferation, differentiation, migration, adhesion and apoptosis.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to or at risk of developing the condition or disorder, as well as those in which the condition or disorder is to be prevented.

As used herein, the term “subject” and “patient” are used interchangeably and refer to a mammal. Mammals include, but are not limited to, domesticated animals (e.g., cows, sheep, cats, dogs, horses, and rabbits), primates (e.g., humans and non-human primates such as monkeys), and rodents (e.g., mice and rats). In certain embodiments, the subject is a human (e.g., a human having a cancer).

### BRIEF DESCRIPTION OF THE DRAWINGS

FIGS. 1A-1F show that Mettl3 and m6A modification on RNA are increased with chemotherapy doxorubicin treatment in MCF7 breast cancer cells and are needed for chemoresistance. FIG. 1A shows Western blot analysis of METTL3, and canonical translation markers, phospho- elF2α and total elF2α in MCF7 cells treated with doxorubicin chemotherapy. FIG. 1B shows dot blots of ribo-minus purified RNA probed for m6A from doxorubicin and control treated cells versus cells depleted of METTL3 with shMETTL3. The Western blot includes samples with control shRNA or shMETTL3. FIG. 1C shows 2D-TLC of ribo-minus purified RNA from doxorubicin treated and untreated cells. m6A and A nucleosides are marked and quantitated. FIG. 1D shows doxorubicin chemosurvival in MCF7 cells expressing (i) control shRNA or shMETTL3, (ii) wildtype METTL3 compared to a control GFP vector, and (iii) m6A catalytically defective METTL3 (CD-M3) compared to wild type METTL3. FIG. 1E shows a Western blot of METTL3 and elF2α that shows increased METTL3 and elF2α phosphorylation increase in patient samples grown in 3D cultures and treated with doxorubicin. FIG. 1F shows a Western blot of METTL3 and elF2α that shows increased METTL3 and elF2α phosphorylation increase in patient-derived xenografts (pdx) that were treated with 2-1 0 mg/kg of doxorubicin in vivo. Tubulin and Histone serve as Western blot loading controls. Data are average of 3 replicates +/-SEM. See also FIGS. 2A-2G.

FIG. 2A shows that increase of METTL3 is a transient stress response and decreases after 24 hr of treatment shown in MCF7 cells. FIG. 2B shows a Western blot with MCF7 cells treated with 500 nM doxorubicin over time. FIG. 2C shows BT549 cells treated with 500 nM doxorubicin over time. FIG. 2D shows THP1 cells treated with 1 µM Cytarabine or AraC over time. FIG. 2E shows 2D-TLC of ribo-minus purified RNA from doxorubicin treated control shRNA cells compared to shMETTL3 cells. Data are average of 3 replicates +/-SEM. Tubulin serves as the loading control. FIGS. 2F and 2G show Western blots of METTL3 and phospho-elF2α that increase over time of treatment with Gemcitabine in MCF7 and BT549 cells (FIG. 2F) or Taxol in BT549 cells (FIG. 2G).

FIGS. 3A-3J show elF2α phosphorylation promotes METTL3 translation. FIG. 3A shows a Western blot of cells treated with poly I:C to induce elF2α phosphorylation. FIG. 3B shows m6A dot blot analysis with poly I:C treatment. FIG. 3C shows treatment of MCF7 cells with Sal003 phosphatase inhibitor to retain elF2α phosphorylation induces METTL3 increase. FIGS. 3D and 3E show METTL3 levels are not increased when doxorubicin treated cells are also co-treated with Trazodone (FIG. 3D) or with ISRIB (FIG. 3E) to override the effect of elF2α phosphorylation as shown by Western analysis. FIG. 3F shows qPCR of METTL3 and METTL14 RNA levels in doxorubicin treated cells, compared to untreated cells. FIG. 3G shows polysome fractionation followed by qPCR analysis below of polysome fractions for METTL3 mRNA and KI67 mRNA in doxorubicin treated cells compared to untreated. FIG. 3H shows polysome fractionation followed by qPCR analysis (graph) of polysome fractions for METTL3 mRNA in Sal003 treated cells compared to untreated cells. FIG. 3I shows Y10B immunoprecipitation from doxorubicin treated cells to verify mRNAs with polysome association, followed by qPCR analysis of METTL3 and METTL14 mRNA as well as control 5.8S rRNA. FIG. 3J shows nascent amino acid labeling and METTL3 immunoprecipitation (Western blot of METTL3 with dark exposure of ⅒ Inputs) from doxorubicin treated versus control cells to detect translation levels (quantitation to the right) of newly labeled immunoprecipitated METTL3 in these cells. Tubulin and Histone serve as Western blot loading controls. Data are average of 3 replicates +/-SEM. See also FIGS. 4A-4H.

FIG. 4A shows Western analyses of MCF7 cells treated with Thapsigargin to induce elF2α phosphorylation. FIG. 4B shows Western analyses of MCF7 cells treated with Torin 1 to block mTORC1 and mTORC2 and induce 4EBP dephosphorylation and elF2α phosphorylation and test METTL3 increase, as well as with mTOR activator MHY1485, to inhibit mTOR and reduce 4EBP dephosphorylation and elF2α phosphorylation FIG. 4C shows Western analyses of MCF7 cells treated with Palomid as another drug to block mTORC1 and mTORC2. FIG. 4D shows Western analyses of MCF7 cells treated with BEZ235 to block PI3K/mTOR and induce 4EBP dephosphorylation and elF2α phosphorylation. FIG. 4E shows m6A dot blot analysis with Torin 1 treatment of 3D tumor spheres where METTL3 and METTL14 increase as shown by Western blot analysis. FIG. 4F shows Y10B immunoprecipitation from Torin 1 treated cells to verify mRNAs with polysome association, followed by qPCR analysis of METTL3 and METTL14 mRNA as well as control 5.8S rRNA. FIG. 4G shows Western blot of nascent amino acid labeling and immunoprecipitation of METTL3 followed by Western analysis of labeling with Streptavidin-HRP and METTL3 to verify immunoprecipitation. FIG. 4H shows 2D-TLC analysis of ribosomal RNA depleted RNA from MCF7 cells treated with buffer or with Sal003 indicating increased m6A (marked) upon treatment with Sal003. Actin and Tubulin are loading controls for Western blots. Data are average of 3 replicates +/-SEM.

FIGS. 5A-5I show HuR and associated translation initiation factors promote METTL3 translation in doxorubicin resistant cells. FIG. 5A shows Western blot of HuR and HuR and Eif3 in eluates after affinity purification by antisense of METTL3 and METTL14 mRNAs compared to a scrambled control, from doxorubicin treated or untreated formaldehyde and UV crosslinked cells. FIG. 5B shows qPCR of METTL3 RNAs co-immunoprecipitated with HuR antibody compared to control IgG. FIG. 5C shows Western blot of HuR and METTL3 in cells transfected with HuR and constitutively cytoplasmic form HuR S221D and mutant HuR-HNS or HuR shRNA. FIG. 5D shows qPCR analysis of METTL3 RNA normalized to Actin RNA upon HuR or control vector overexpression in doxorubicin treated cells. FIG. 5E shows overexpression of HuR compared to control followed by doxorubicin treatment to test chemotherapy survival shown by percentage surviving cell count. FIG. 5F shows HuR immunoprecipitates co-purifies elF2D and elF3a as well as METTL3 mRNA and METTL3 5′UTR (side graph) from doxorubicin treated cells but not untreated cells after in vivo UV crosslinking, as detected by RT-qPCR. FIG. 5F also shows Western blot of the interaction of HuR and elF3a with elF2D upon elF2D immunoprecipitation that was analyzed by qPCR for co-purification of METTL3 mRNA in doxorubicin treated and untreated cells. FIG. 5G shows co-immunoprecipitation of METTL3 mRNA with elF2D compared to IgG control in doxorubicin treated (left graph) compared to untreated cells (right graph). FIG. 5H shows luciferase activity (relative light units, RLU) of Firefly luciferase reporter bearing the 5′UTR of METTL3 normalized to co-transfected Renilla in untreated and doxorubicin treated cells. FIG. 5I shows luciferase activity (relative light units, RLU) of a mutated GAC site upstream of the reporter start site in the 5′UTR of METTL3 normalized to co-transfected Renilla compared to control UTR reporter in doxorubicin treated cells. Tubulin is the loading control for Western blots. Data are average of 3 replicates +/-SEM. See also FIGS. 6A-6K.

FIG. 6A shows increase of HuR with doxorubicin treatment (one of the RNA binding proteins and translation factors that increase on doxorubicin treatment from mass spectrometry analysis). The Western blot shows HuR increase over time of doxorubicin treatment. FIG. 6B shows Mettl3 and Mettl14 mRNA antisense purification of RNA binding proteins and translation factors from formaldehyde crosslinked extracts of doxorubicin treated or untreated cells compared to a scrambled antisense. Western blot is shown of other RNA binding proteins that increase in mass spectrometry data. FIG. 6C shows Relative Luciferase activity of Firefly Luciferase reporter bearing the 3′UTR of METTL3 normalized to a Renilla control reporter shows no increase in doxorubicin treated cells compared to untreated cells. FIG. 6D shows HEXIM1 increases along with METTL3 in doxorubicin treated cells. FIG. 6E shows HMBA treatment that increases HEXIM1 increases METTL3 and METTL14 along with elF2α phosphorylation. FIG. 6F shows Western blot of Hexim1 and METTL3 and qPCR of METTL3 RNA normalized to Actin RNA in cells transfected with two shRNAs against Hexim1. FIG. 6G shows Western blot of Hexim1 immunoprecipitates that was analyzed by qPCR for co-purification of METTL3, METTL14 mRNAs and tRNA-Met (graphs below) in doxorubicin treated and untreated cells. FIG. 6H shows translation initiation factor elF2β immunoprecipitation followed by Western analyses of Hexim1 and elF5B. FIG. 6I shows Y10B antibody immunoprecipitation to detect polysome associations followed by Western analysis of Hexim 1 and qPCR analysis of 5.8S rRNA as control for Y10B purification of polysomes. FIGS. 6J and 6K show Hexim, PPM1G and HuR increase in chemotreated patient samples and on doxorubicin treatment.

FIGS. 7A-7F show METTL3 promotes chemoresistance by suppressing proliferation and antiviral response, while promoting invasion genes. FIG. 7A shows m6A IP genes from doxorubicin treated cells but not in untreated cells (2335) were compared with genes upregulated in shMETTL3 cells versus control shRNA cells (5168) to identify genes that are m6A marked and downregulated by METTL3 (Venn Diagram) (i). Upregulated genes were also compared but were fewer. FIG. 7A also shows genes that are m6A associated: cell cycle genes (Ki67, PLK1), and antiviral (DDX58 or RIG-I, PKR) genes but not control genes such as Actin and tRNA-lys are associated with m6A antibody and are increased upon METTL3 depletion in shMETTL3 cells at the protein and RNA levels by TMT mass spectrometry and microarray (ii). FIG. 7A further shows GSEA analysis of m6A associated genes reveals enrichment of cell cycle genes that are significantly (>=1.5 fold) upregulated upon METTL3 depletion (iii). FIG. 7B shows Western blot analysis of RIG-I antiviral protein in shMETTL3 compared to control shRNA expressing doxorubicin treated cells (i). FIG. 7B also shows M6A antibody immunoprecipitation of RIG-I and PKR antiviral gene mRNAs compared to IgG control (ii). FIG. 7B further shows an invasion assay performed on Matrigel transwell plates with MCF7 control shRNA cells or with METTL3 depleted cells. The number of invading cells normalized for the total number of cells plated are shown (iii). FIG. 7C shows antiviral response to treatment with poly I: C was tested by qPCR levels of RIG-I and PKR RNAs. This was tested in METTL3 overexpression cells compared to CD-M3 cells compared to control Actin mRNA by qPCR. Shown Western blot of METTL3 levels in cells with METTL3, METTL14 and CD-M3 overexpression and METTL3 depletion compared to control vector. FIG. 7D shows cell adhesion and invasion genes are upregulated by METTL3, as decreased cell adhesion and invasion genes are observed with METTL3 depletion. FIG. 7E shows downstream targets of RIG-I, CASP9 and STAT1, are increased in METTL3 depleted cells, as observed by qPCR analysis normalized for tRNA-lys. FIG. 7F shows inhibition of doxorubicin chemosurvival with inhibitors that override downstream of elF2α phosphorylation and integrated stress response (trazodone, ISRIB), compared to buffer treated control. 500 nM Doxorubicin treated MCF7 cells were co-treated with (i) 5 uM of Trazodone, (ii) 5 uM of ISRIB, or DMSO buffer. Western blot analyses of METTL14 levels and elF2α phosphorylation are shown in FIG. 5A compared to buffer treated control cells. Data are average of 3 replicates +/-SEM. Actin and Tubulin are loading controls for Western blots. See also FIG. 8A-8l.

FIG. 8A shows doxorubicin treated control shRNA cells compared with shMETTL3 cells (percentage chemosurviving cells) after treatment with buffer or Trazodone to override the elF2 phosphorylation pathway and block the METTL3/14 induced chemoresistance to test whether the chemoresistance effect bypassed by Trazodone was due to METTL3. Treatment of shMETTL3 cells with doxorubicin and with Trazodone that overrides elFα2 phosphorylation and integrated stress response, does not cause additional loss of chemosensitivity, indicating that METTL3 and the target of this inhibitor are in the same pathway and that the reduced chemoresistance with Trazodone or ISRIB in FIG. 7F is due to reduced METTL3. FIG. 8B shows survival of MCF7 cells with Doxorubicin treatment versus doxorubicin and BMN-673 PARP inhibitor treatment. FIGS. 8C and 8D show Western blot analysis of PARP1, phospho-elF2α, METTL3 with doxorubicin or Sal003 treatment. FIG. 8E shows genes that are upregulated in doxorubicin proteome and are m6A immunoprecipitated, including PARP1, ADAR, APOBEC3B and METTL3 itself. FIG. 8F shows METTL3 decreases with reduction of elF2α phosphorylation with treatment with metformin or phenformin. FIG. 8G shows inhibition of doxorubicin chemosurvival with inhibitors of PKC that activates elF2α phosphorylation and HuR (enzastaurin), compared to buffer treated control. 500 nM Doxorubicin treated MCF7 cells were co-treated with 6uM of Enzastaurin or DMSO buffer. FIG. 8H shows Western analyses of doxorubicin treated cells that were co-treated with drugs to override PKC (Enzastaurin) or elF2α phosphorylation (ISRIB) followed by analyses of METTL3 and METTL14. Co-treatment with doxorubicin and Sal-003 reduces the effect while only Sal-003 treatment retains elF2α phosphorylation and METTL3 and METTL14 levels in FIGS. 3A-3J. FIG. 8I shows Western analysis after p38 MAPK inhibitor (SB=SB203580, LY2228820) reduces METTL3 increase. Data are average of 3 replicates +/-SEM.

FIG. 9 shows a flowchart of a chemotherapy-induced stress response in cancer cells and various inhibitors or overriders useful for treating chemosurival.

### DETAILED DESCRIPTION OF THE INVENTION

Chemotherapy-induced stress leads to downregulation of canonical translation in quiescent (G0) cells, which enables alternate post-transcriptional mechanisms that enable chemosurvival. Modifications on RNAs have been recently shown to cause their post-transcriptional regulation in distinct cellular conditions. These alter structure, or mRNA and protein interactions, and recruit RNA binding proteins called readers that recognize the modification on mRNAs to cause post-transcriptional regulation of such mRNAs. Deregulation of the RNA methyltransferases or writers, their RNA binding protein effectors (readers) or their demethylases (erasers) have been implicated in various diseases including cancer. Such modifications also mark cellular RNAs as self to avoid triggering the cellular anti-viral response. The m6A RNA methyltransferase, methyltransferase-like 3 (METTL3), associates with its co-factor methyltransferase-like 14 (METTL14), to methylate the N6 position of Adenosine on mRNAs at RRACH motifs (in which R represents A or G, and H represents A, C or U). METTL3 and METTL14 have been implicated in the control of the embryonic stem cell state, in stress conditions, and in cancers where their expression is deregulated, causing disease by altering m6A target mRNA gene expression via RNA stability or translation changes.

Here, we found that the m6A RNA methyltransferase, METTL3, increases transiently, along with increased elF2α phosphorylation in doxorubicin and other chemotherapy-surviving cells in vitro and in vivo, enhancing m6A on RNA. elF2α phosphorylation and mTOR inhibition that also induces elF2α phosphorylation decrease canonical translation in these cells, permitting non-canonical translation of METTL3 and METTL14. Consistently, integrated stress response activator and elF2α phosphatase inhibitor promote METTL3. METTL3 translation requires RNA binding proteins and non-canonical translation factors that are enabled by therapy induced elF2α phosphorylation. METTL3 RNA affinity purification reveals elF3a and HuR that interact with elF2D and promote METTL3-5′UTR translation that is enhanced on METTL3 depletion.

Further, METTL3 downregulation reduces proliferation and antiviral-immune-response genes, while promoting DNA repair enzymes, such as poly adenosine diphosphate-ribose polymerase 1 (PARP1), and DNA-RNA editing enzymes, such as adenosine deaminases acting on ribonucleic acid (ADAR) and Apolipoprotein B MRNA Editing Enzyme Catalytic Subunit 3B (APOBEC3B); consistently, METTL3 depletion or overriding phospho-elF2α or such genes reduces chemosurvival. Our data reveal that m6A-mediated gene expression regulation increased chemosurvival and depleting METTL3 or overriding elF2α phosphorylation that induces METTL3 in chemotherapy treated cells, reduced chemosurvival. These data reveal that stress signals promote non-canonical translation of the m6A enzyme METTL3 that controls gene expression for therapy survival.

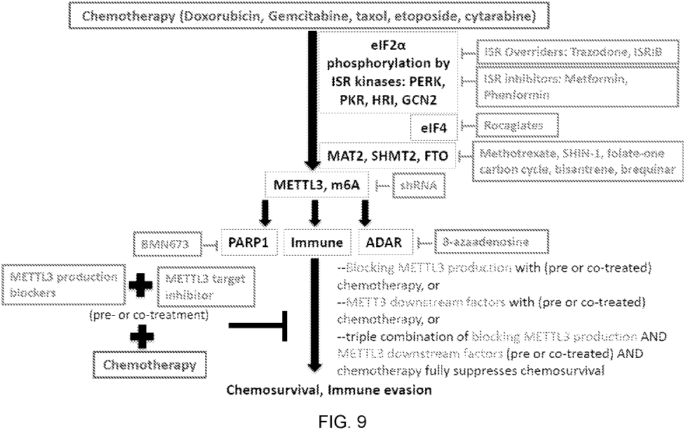

Referring to FIG. 9, we describe an overview of the points of regulation of METTL3 and m6A in view of our findings. For example, chemotherapy (e.g., doxorubicin, gemcitabine, taxol, etoposide, or cytarabine) in cancer cells, such as acute myeloid leukemia cells or breast cancer cells (e.g., triple negative breast cancer cells or hormone-positive breast cancer cells), induces the integrated stress response (ISR). The ISR involves four kinases: PERK, PKR, HRI, and GCN2. Induction of the ISR increases METTL3, which involves elF4, serine hydroxymethyltransferase 2 (SHMT2), methyl adenosyltransferase 2 (MAT2 (e.g., MAT2A and MAT2B)), and fat mass and obesity-associated (FTO) demethylase. METTL3 modifies RNA with m6A that suppresses cell cycle genes and antiviral genes and increases ADAR, PARP1, and immune modulators that lead to chemosurvival and immune evasion. To block METTL3 production, the ISR can be overridden upstream of METTL3 by trazodone or integrated stress response inhibitor (ISRIB) or inhibited by metformin or phenformin. Furthermore, METTL3 mRNA is bound by proteins that are needed for increased METTL3 and m6A. These include FTO along with elF4, SHMT2, and MAT2A and MAT2B, proteins that bind METTL3 in our in vivo crosslinked affinity purification and regulate its expression via its GAC 5′UTR. elF4 can be targeted with rocaglates; SHMT2 and MAT2 can be targeted with folate/one-carbon metabolism inhibitors, Serine hydroxymethyltranferase inhibitor 1 (SHIN-1), or methotrexate; and FTO demethylase can be targeted with bisantrene or brequinar to block METTL3 production upstream of METTL3. METTL3 production can also be directly blocked by, for example, short hairpin RNA (shRNA) or small interfering RNA (siRNA). Downstream factors of METTL3 can also be targeted to inhibit METTL3. PARP1 can be inhibited by BMN673 (talazoparib), and ADAR can be inhibited by 8-azaadenosine. A combination of a chemotherapeutic with an agent that blocks METTL3 production and/or an agent that inhibits METTL3 downstream factors can suppress chemosurvival.

We now describe the results of our studies.

### Results

### METTL3 and m6A on RNA Increase in MCF7 and BT549 Breast Cancer Cells Treated with Doxorubicin Chemotherapy

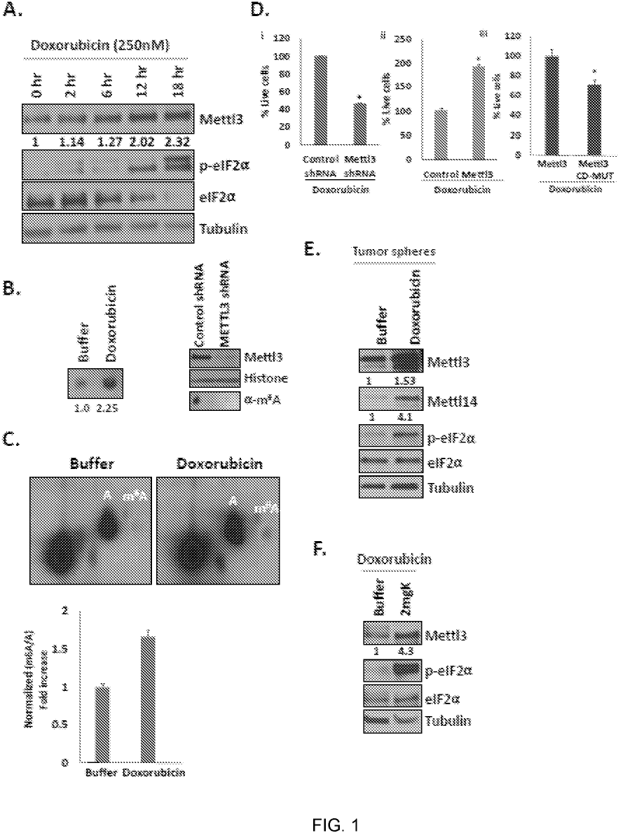

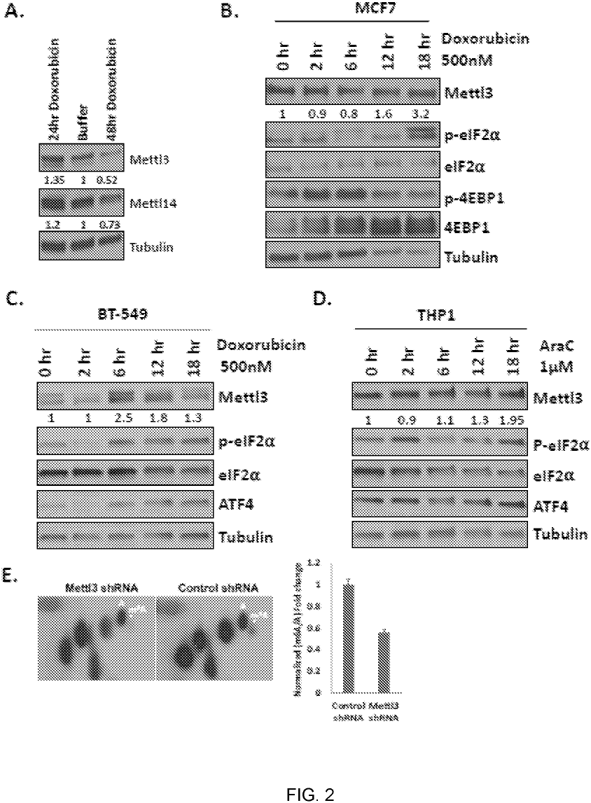

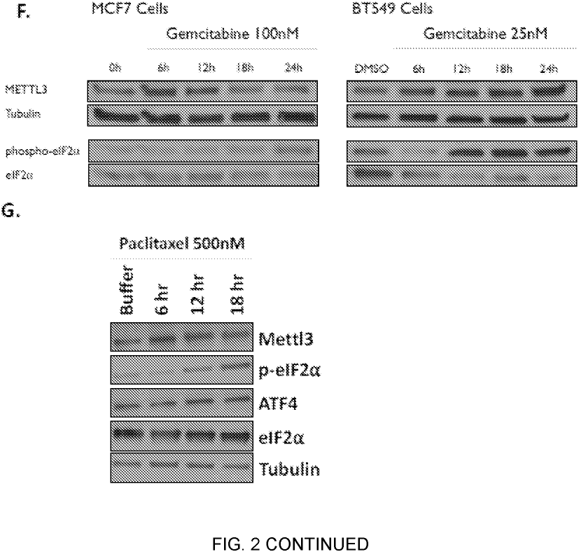

Our data revealed that canonical post-transcriptional mechanisms are altered in G0, chemosurviving cancer cells and are replaced by other distinct mechanisms. To determine the mechanisms of specific mRNA expression in chemosurviving cancer cells, we examined RNA binding regulators in chemosurviving MCF7 breast cancer cells, isolated after doxorubicin chemotherapy treatment. Under these conditions, elF2α is phosphorylated over time of doxorubicin addition. Concurrently, we found that the RNA m6A methyltransferase METTL3 increases in chemosurviving MCF7 cells treated with doxorubicin chemotherapy (FIG. 1A). Increase in METTL3 was observed with doxorubicin treatment (500 nM) transiently over time (FIG. 2B). This increase was not unique to MCF7 cells, as multiple concentrations tested on triple negative breast cancer BT549 cells or acute monocytic leukemic THP1 cells treated with another chemotherapy used in leukemia, Cytarabine (AraC), revealed similar transient increase in elF2α phosphorylation and METTL3 (FIGS. 2C and 2D). The increase observed is transient and is not observed at 48 h of treatment (FIG. 2A). These data showed that METTL3 increased as a stress response transiently, along with elF2α phosphorylation.

To test whether the increased m6A modification enzymes lead to increased m6A modification on RNA, ribosomal RNA depleted RNA from doxorubicin treated cells compared to untreated cells by dot blot analyses was tested. Consistent with the increased METTL3 levels (FIGS. 1A and 2B-2D), doxorubicin treated cells showed more m6A modified RNA compared to untreated cells (FIG. 1B). To verify these results, first, a cell line was engineered using standard methods with a constitutively expressed shRNA that depletes METTL3 and dot blot analysis was performed. Consistent with decreased METTL3 upon knockdown, the dot blot signal is depleted when METTL3 is knocked down (FIG. 1B). Second, RNA from doxorubicin treated cells for m6A by two-dimensional-thin layer chromatography separation (2D-TLC) was performed (H. Grosjean, G. Keith, L. Droogmans, Detection and quantification of modified nucleotides in RNA using thin-layer chromatography. Methods in molecular biology (Clifton, N.J.) 265, 357-391 (2004)). We found that the m6A signal on 2D-TLC increased in ribosomal RNA depleted, poly(A) selected RNAs from doxorubicin treated cells compared to untreated cells (FIG. 1C); this signal is reduced in RNA from shMETTL3 expressing cells compared to control shRNA cells (FIG. 2E), verifying the m6A signal. These data indicate that the increased METTL3 leads to increased m6A on RNA in doxorubicin treated cells.

### METTL3 is Required for Chemosurvival and Increases with Doxorubicin Treatment in Patient Samples and in Patient Derived Xenografts

As METTL3 increases in chemosurviving cells and increases m6A on RNA, we tested whether METTL3 increase is needed for chemosurvival by depleting METTL3 or overexpressing METTL3 or a catalytically defective METTL3 (CD-M3) mutant and then tested these cells for doxorubicin survival. We found that compared to a control shRNA, stable cell lines that deplete METTL3 (FIG. 1B), showed decreased chemosurvival (FIG. 1D). Correspondingly, overexpression of METTL3 showed increased chemoresistance while overexpression of the m6A defective mutant did not (FIG. 1D). These data indicated that METTL3 transiently increases in chemotherapy treated cells, causing increased m6A on RNA that leads to altered gene expression that contribute to chemosurvival of such cells. Consistently, we found that METTL3 is increased in hormone positive breast cancer patient samples grown as tumor spheres and treated with doxorubicin (FIG. 1E). Increase of METTL14 was also observed with doxorubicin where METTL3 is also increased (FIG. 1E). Furthermore, we found that METTL3 also increases in in vivo samples, in doxorubicin treated hormone positive breast cancer patient derived xenografts (FIG. 1F). These data evidenced that the increase in METTL3 observed in chemotherapy treated cell lines is not an artifact as METTL3 increases in chemosurviving primary breast cancer samples.

### METTL3-METTL14 and m6A Increase with Poly I:C Induction of ISR

Given that the increase of METTL3 was observed with multiple chemotherapies including DNA damage drugs such as doxorubicin, AraC, Gemcitabine as well as mitotic inhibitors such as Taxol (FIGS. 2B, 2C, 2F, and 2G), it showed that METTL3 increased in response to stress signals. Canonical translation is suppressed in G0 chemosurviving cells, via inhibition of elF2α and cap dependent translation.

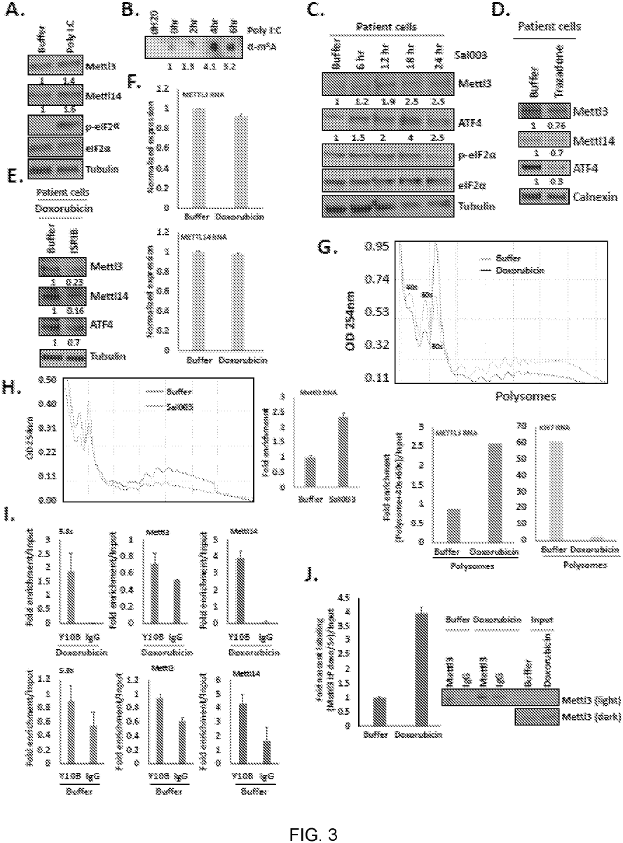

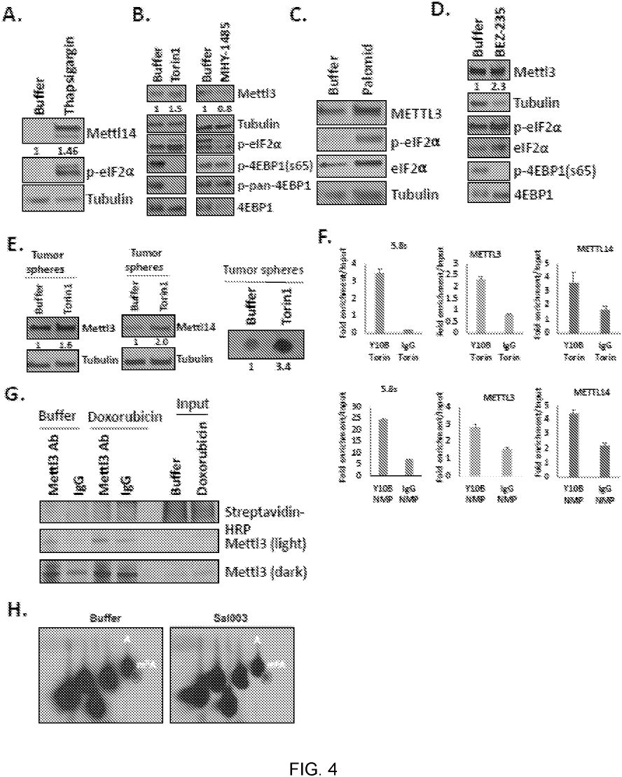

Doxorubicin chemotherapy treatment in breast cancer cells as well as other chemotherapies like AraC in THP1 cells, can promote elF2α phosphorylation that correlated with METTL3 increase, as shown in FIGS. 1A and 2B-2D. This was also observed in patient samples and in vivo (FIGS. 1E and 1F). elF2α phosphorylation is triggered by ISR, which can be activated by double-strand RNA mimic, poly I:C or by thapsigargin; consistently, we found that thapsigargin or poly I:C induces METTL3 and METTL14 increase along with elF2α phosphorylation (FIGS. 3A and 4A); consistently, we found increased m6A signal on dot blots of ribosomal RNA depleted RNAs derived from poly I:C treated cells (FIG. 3B) where METTL3 increases. These data indicate concurrent upregulation of METTL3 with induction of elF2α phosphorylation.

### METTL3-METTL14 and m6A Increase with Induction of elF2α Phosphorylation Observed Upon Treatment with mTOR Inhibitors

In cells treated with chemotherapy such as doxorubicin, ISR kinases, including PKR and PERK that phosphorylate elF2α, are transiently activated. Such cells also show mTOR/PI3K inhibition can activate PKR similar to poly I:C treatment and mTOR inhibition can lead to elF2α phosphorylation. Therefore, we treated MCF7 cells with Torin1 that blocks both mTORC1 and mTORC2 to test whether inhibition of mTORC1 and mTORC2, would activate the pathway that promotes METTL3.

As shown in FIG. 4B, we found that upon Torin1 inhibition of mTORC1 and mTORC2, METTL3 increased in MCF7 cells along with elF2α phosphorylation; 4EBP dephosphorylation marked the efficacy of Torin 1. In contrast, mTOR activation with MHY1485 decreased METTL3. Increase in METTL3 was also observed with Palomid 529, an mTORC1 and mTORC2 inhibitor (FIG. 4C) and with BEZ 235, a dual inhibitor (FIG. 4D) of mTOR and PI3K that prevents reactivation of mTOR. METTL14 and METTL3 increase (FIG. 4E) were also observed in Torin1 treated 3D tumor spheres. Concordantly, increased m6A on RNA (FIG. 4E) was observed by dot blot analyses in torin 1 treated MCF7 3D tumor spheres that mimic in vivo tumors (T. Muranen et al., Inhibition of PI3K/mTOR leads to adaptive resistance in matrix-attached cancer cells. Cancer cell 21, 227-239 (2012)). Thus, we found that mTOR inhibitors torin1 or BEZ 235 treatments, but not mTOR activator MHY1485, show increased METTL3 along with elF2α phosphorylation (FIGS. 4B-4E).

### Induction of elF2α Phosphorylation Increases METTL3 and METTL14

From the above data, the common feature of all these conditions that promote METTL3 increase is the associated increase in elF2α phosphorylation. To test the need for elF2α phosphorylation for the increase of METTL3, we used an established inhibitor of elF2α phosphatase, Sal003, that would maintain increased elF2α phosphorylation levels. As shown in FIG. 3C, we found that Sal003 treatment prevented dephosphorylation of elF2α and induced the known non-canonical translation of ATF4 that is associated with increased levels of elF2α phosphorylation; consistently, Sal003 increased METTL3 and METTL14 levels in MCF7 cells. If METTL3 is increased by Sal003, then m6A on RNA should increase in such conditions. These data showed that induction of elF2α phosphorylation leads to the increase of METTL3.

elF2α phosphorylation pathway can be bypassed pharmacologically using drugs that override the effect of elF2α phosphorylation: trazodone that is used clinically as an anti-depressant is thought to override elF2α phosphorylation at the ternary complex stage downstream or ISRIB, which overrides the effect of elF2α phosphorylation by affecting elF2B, the Guanine nucleotide exchange factor for elF2α. We co-treated cells with doxorubicin and with or without these ISR inhibitors that override elF2α phosphorylation. As a control, we checked the levels of ATF4, an ISR response gene that is increased by non-canonical translation due to elF2α phosphorylation. As shown in FIGS. 3D and 3E, we found that doxorubicin treated cells that are also treated with either trazodone or ISRIB, do not show increase of ATF4, and consistently, also do not show increase of METTL3 levels, compared to buffer treated cells. These data implicate the elF2α phosphorylation pathway in promoting METTL3 levels by non-canonical translation in doxorubicin surviving cells. These data evidenced that chemotherapy stress signaling activates elF2α phosphorylation to promote METTL3 levels via non-canonical translation.

### METTL3 mRNA is Increased on Polysomes and METTL3 Nascent Translation is Increased in Doxorubicin Treated Cells

As elF2α phosphorylation alters translation, these data showed that METTL3 is increased via non-canonical translation in these conditions. Consistently, no increase of METTL3 and METTL14 was seen at the RNA levels by qPCR analyses in doxorubicin treated cells in FIG. 3F, further supporting that METTL3 and METTL14 are increased at the translation or post-translation levels. To verify that the increase in METTL3 was at the translation level, polysome analysis was conducted first. We found that METTL3 mRNA increased on polysomes compared to monosomes in doxorubicin treated cells compared to buffer treated cells; in contrast, the cell cycle marker KI67 mRNA that is not translated in these conditions decreased on polysomes (FIG. 3G). To show that the translation effect on METTL3 was induced by elF2α phosphorylation, polysome analysis was performed with Sal-003 phosphatase inhibitor. As shown in FIG. 3H, METTL3 mRNA increased on polysomes compared to monosomes in Sal-003 treated cells compared to buffer treated cells, showing that METTL3 mRNA is translated in conditions of elF2α phosphorylation. Second, Y10B antibody was previously demonstrated to bind 5.8S rRNA in assembled ribosomes and has been used to show ribosome association. We next verified increased polysome association of METTL3 mRNA with Y10B antibody immunoprecipitation in doxorubicin treated and in Sal003 cells compared to buffer treated cells (FIGS. 3I and 4F). Previous data had revealed that METTL3 and METTL14 are co-regulated; consistently, we observe that METTL14 is regulated similarly without RNA level change and is associated with Y10B (FIGS. 3I and 4F). Third, to ensure that these associations are not indirect and METTL3 increase is due to enhanced translation, we used an in vivo nascent translation assay by amino acid labeling (Click-it Puro) to test whether more newly translated METTL3 can be detected in doxorubicin treated cells. As shown in FIGS. 3J and 4G, immunoprecipitation of METTL3 from labeled cells revealed an increase of labeled METTL3 in doxorubicin treated cells compared to untreated cells, indicating increased translation of the protein. These data indicate that METTL3 mRNA is translationally upregulated in doxorubicin-resistant cells that show elF2α phosphorylation.

### RNA Binding and Translation Factors Associate with METTL3 mRNA

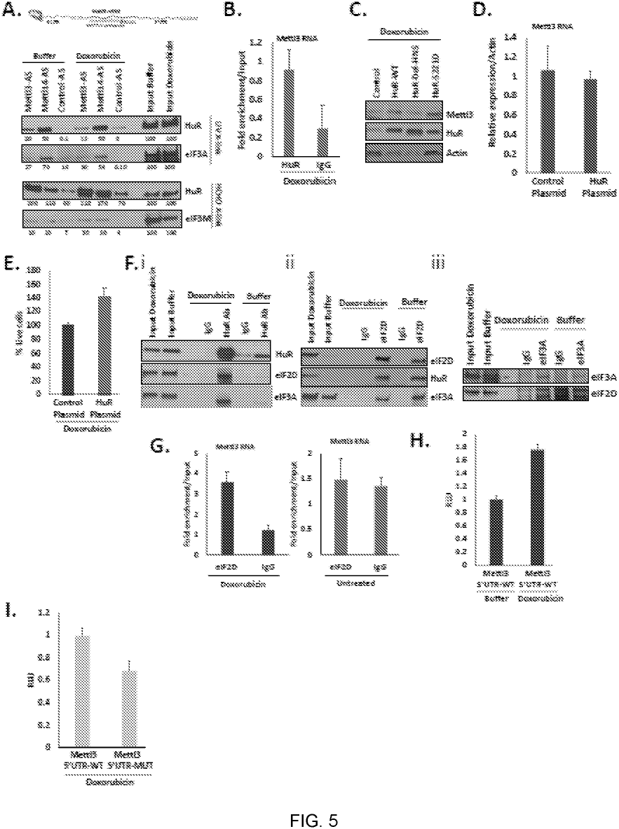

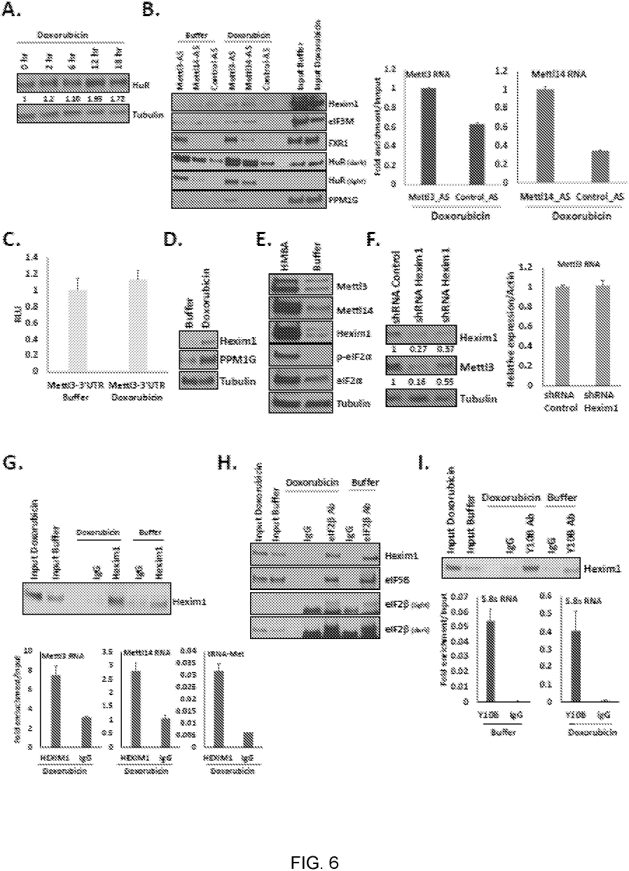

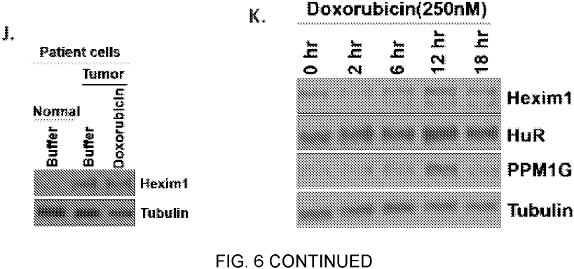

To identify proteins that associate with METTL3 mRNA to promote its non-canonical translation, we used a biotin tag antisense to METTL3 and METTL14 mRNAs, against shRNA target sites, as these sites are verified to be available for base pairing with the antisense, compared to a scrambled control antisense. We tested the antisense purification for association with RNA binding proteins and translation factors that are specifically increased in chemosurviving cells, identified by Tandem-Mass-Tag (TMT) spectrometry (FIG. 6A). Purification of METTL3 and METTL14 RNAs from in vivo formaldehyde crosslinked extracts from doxorubicin treated cells (FIGS. 5A and 6B) revealed that METTL3 and METTL14 mRNAs but not a control antisense, associates with several RNA binding proteins that increase in these conditions. We identified HuR and elF3a, and to a lesser extent other proteins that are also increased in these stress conditions, Hexim1 and its associated factor, PPM1G (FIG. 6B). In extracts that were UV crosslinked to look for direct binding, we found HuR and elF3a associate with METTL3 mRNA by RNA antisense affinity purification. METTL14 mRNA antisense associated with these factors also, indicating co-regulation that has been noted. Consistently, this coregulation is observed in our proteomic data where METTL14 decreases upon METTL3 depletion. Together, METTL3 mRNA associates with HuR and with translation factors such as elF3A in doxorubicin treated cells.

### HuR Associates with METTL3 mRNA and Promotes METTL3 Increase in Doxorubicin Surviving Cells; and Consistently, Promotes Chemosurvival

To test the role of HuR, we first depleted HuR or overexpressed HuR that is cytoplasmic (S221D). We found that HuR depletion reduces METTL3 levels without affecting its RNA levels while overexpression of HuR-S221D that has a constitutive phosphorylation form that is cytoplasmic, promotes METTL3 (FIG. 5B). We found that while HuR overexpression promotes METTL3 levels, it does not increase METTL3 RNA levels (FIG. 5C), evidencing that HuR overexpression impacts METTL3 increase via translation regulation. Consistently, depletion of HuR decreases METTL3 levels (FIG. 5D). These data indicate that HuR promotes METTL3 mRNA translation.

METTL3 overexpression promotes chemosurvival and increases in chemoresistant cells where it is needed for chemosurvival (FIGS. 1A, D). Therefore, overexpression of HuR that promotes METTL3 levels, should also promote chemosurvival. We found that HuR overexpressing cells do promote chemosurvival when treated with doxorubicin (FIG. 5E). Together, these data showed that HuR binds METTL3 mRNA to promotes its translation and thereby promote chemosurvival in chemotherapy treated cells where HuR increases.

### HuR Associates with eIF3a and eIF2D That is Needed for METTL3 Translation in Doxorubicin Resistant Cells and Upon elF2α Phosphorylation

To understand how HuR promotes translation along with the factors, elF3a and elF2D, we first conducted immunoprecipitation to test whether they associate and with METTL3 mRNA and looked for translation factors. We first confirmed association with HuR by in vivo crosslinking coupled co-immunoprecipitation of METTL3 mRNA with HuR antibody. We found that HuR co-immunoprecipitates METTL3 mRNA (FIG. 5F), affirming the interaction with the mRNA. We found that HuR associates with a non-canonical translation factor and tRNA recruiter, elF2D, and both proteins associate with elF3a, the other factor that associated in in vivo UV crosslinked samples with METTL3 mRNA antisense (FIG. 5G). elF2D is an alternate translation initiation factor that can bring in the tRNA at all cognate and non-cognate initiation sites when elF2α is phosphorylated. eIF2D also immunoprecipitated both HuR and elF3a. Both HuR and elF2D interact, and elF2D co-immunopurify METTL3 mRNA in doxorubicin treated cells where elF2α is phosphorylated but not in untreated cells (FIGS. 5F and 5H). EIF3 is a multiprotein complex including elF3a that can recruit the ribosome for translation and its components are known to initiate non-canonical translation of specific mRNAs. Consistently, we found that depletion of elF3a reduced METTL3 levels, while overexpression of elF3a promoted METTL3 levels and accordingly, enhanced chemosurvival. These data showed that HuR and elF3a associate with METTL3 mRNA to promote its translation involving non-canonical translation factor elF2D as well as elF3a itself.

We depleted elF2D to test its impact on METTL3 levels and translation. We found depletion of elF2D prevented increase of METTL3 levels in doxorubicin treated cells, consistent with a role in promoting its non-canonical translation via association with METTL3 mRNA and METTL3 mRNA associated factors such as HuR and reduced chemosurvival (FIGS. 5A, 5B, 5F, and 5G). These data showed that upon elF2α phosphorylation, elF2D promotes non-canonical translation of specific mRNAs like METTL3 to which it is associated along with factors such as HuR and elF3a. Depletion of elF2D along with overexpression of HuR did not enable METTL3 increase, indicating that HuR needed elF2D to promote METTL3 translation.

### HEXIM1, PPM1G and Translation Factors Associate with METTL3 mRNA and HuR to Regulate METTL3 Levels

Other RNA binding proteins such as HEXIM1 and PPM1G that increase in doxorubicin surviving cells also associate with METTL3 mRNA and with HuR. The indirect associated protein, Hexim1 is a transcription regulator but also associates with other complexes and RNAs and promotes mRNA stability under nucleotide stress. Hexim1 increases with poly I:C that promote METTL14, in chemoresistant patient sample, and in mTOR inhibited conditions that increases elF2α phosphorylation, METTL3 and METTL14 (FIG. 6D). The phosphatase PPM1G is known to associate with Hexim1 and is activated by DNA damage/ATM signaling upon chemotherapy treatment, and upon PI3K/Akt inhibition, consistent with these conditions where METTL3 increases. On Akt inhibition, PPM1G dephosphorylates 4EBP; this inhibits canonical translation, consistent with specific mRNA translation elicited in these conditions.

Hexim1 is induced by HMBA treatment that can activate PKC and phosphorylate Hexim1; consistently, we found that HMBA increased Hexim1 as well as METTL3 (FIG. 6E). Therefore, we tested whether Hexim1 was required for METTL3 levels. Hexim1 protein depletion reduced METTL3 protein but did not affect METTL3 RNA levels, indicating that Hexim1 is involved in regulating METTL3 protein levels (FIG. 6F). Immunoprecipitation of Hexim1 revealed association of METTL3 and METTL14 mRNAs, as well as of tRNAs including tRNA-Met, in doxorubicin treated cells but not in untreated proliferating cells (FIG. 6G). However, HEXIM1 and PPM1G did not bind METTL3 mRNA in UV crosslinked cells (not shown), indicating that the association is indirect, through association with HuR (FIG. 6F). We found that HEXIM1 associates with elF2β that binds tRNA-Met, and with the translation initiation factor, eIF5B that can support non-canonical translation in elF2α phosphorylation conditions, indicating association with the translation machinery (FIG. 6H). Consistently, in vivo formaldehyde crosslinking and HEXIM1 immunoprecipitation followed by TMT-mass spectrometry revealed that HEXIM1 associated with not only HuR and PPM1G, but also several translation initiation factors including elF3M, elF2β, PABPN, and cap binding proteins NCBP1 and NCBP3 as well elF3D. These data reveal HEXIM1 as an RNA binding protein, increased in stress induced conditions that cause elF2α phosphorylation, and associates with the translation machinery and METTL3 and METTL14 mRNAs. Consistently, we found that Hexim1 associates with Y10B antibody, indicating HEXIM1 association with ribosomes (FIG. 6I). These data indicated that Hexim1 could indirectly associate with and promote METTL3 and METTL14 levels in these conditions.

### METTL3 5′UTR Promotes Reporter Translation in Response to Doxorubicin and Sal003 Treatment

To test if METTL3 mRNA encodes for cis-acting elements that direct its translation upregulation in doxorubicin-treated cells, we constructed Luciferase reporters bearing METTL3 mRNA 5′UTR and 3′UTR, or their reverse sequences or vector sequence as controls. We tested these reporters and their controls in untreated and doxorubicin treated cells. We found that METTL3 5′UTR reporter promoted translation over 3-fold compared to both vector and reverse UTR control reporters. Critically the 5′UTR reporter enhanced translation in doxorubicin treated cells compared to untreated cells, mimicking the endogenous METTL3 (FIG. 5H). The 3′UTR reporter did not upregulate translation compared to the vector control or when comparing untreated and doxorubicin treated cells, indicating that while the 3′UTR or its reverse sequence affects expression (FIG. 6C), it was not involved in the translation upregulation on doxorubicin treatment. Importantly, RNA levels of the 5′UTR reporter remained unaffected between doxorubicin treated compared to untreated cells, and compared to reverse or vector reporters, indicating translation control. In vivo crosslinking coupled co-immunoprecipitation revealed association of HuR with the 5′UTR and not the 3′UTR of METTL3, evidencing the 5′UTR was involved (FIG. 5F). These data indicate that METTL3 5′UTR imparts the code for translation upregulation upon doxorubicin treatment.

### M6A Site GAC in METTL3 5′UTR and METTL3 Levels Regulate Translation

While m6A modification causes RNA downregulation, it can also promote non-canonical translation in the 5′UTR via elF3a recruitment to the m6A modified GAC motif. HuR can also bind m6A modified sites to protect from RNA decay and is known to regulate UTR dependent translation. We identified that the METTL3 mRNA bound complex includes both HuR and elF3A, showing that m6A modification mediated translation regulation is happening on METTL3 mRNA that harbor GAC motifs within an RRACH region upstream and downstream of the ATG. Therefore, we constructed reporters with METTL3 5′UTR with the GAC sites mutated. We found that while the 5′UTR reporter increased in doxorubicin treated cells compared to untreated cells, the GAC mutant reporter difference was not significant (FIG. 5I). These data evidence that METTL3 mRNA is regulated in doxorubicin compared to untreated cells via a GAC motif in its 5′UTR.

We also found that the GAC motif mutant reporter translated better than the wild type 5′UTR reporter in untreated conditions, indicating that the m6A site is repressive in untreated conditions; the GAC motif reporter was not significantly different between untreated and doxorubicin treated cells (FIG. 5I). This showed that the presence of METTL3 levels or activity regulates its own 5′UTR reporter translation. To verify the role of METTL3 levels on its 5′UTR reporter translation, we expressed METTL3 5′UTR luciferase reporter in cells with and without overexpression of METTL3 and upon its depletion. METTL3 overexpression reduced 5′UTR reporter translation compared to a control. These results were not observed with a control 5′UTR luciferase reporter or with the mutated GAC METTL3 5′UTR luciferase reporter. The impact on 5′UTR reporter translation by METTL3 overexpression or CD-mutant was not significant, indicating a role for METTL3 levels via its function on translation rather its catalytic activity. Consistently, depletion of METTL3 increased translation of the UTR reporter, compared to control shRNA depletion in both untreated and doxorubicin treated cells, indicating that the reporters show more translation when METTL3 levels are low. These data indicate that METTL3 levels are mediated by METTL3 regulation of its 5′UTR dependent translation. The m6A motif that serves as a binding site is required for doxorubicin responsive upregulation where METTL3 mediated repression is impaired to increase METTL3 levels until sufficient levels are reached. This is due to its recognition by FTO along with elF4, SHMT2, and MAT2A and MAT2B, proteins that bind METTL3 in our in vivo crosslinked affinity purification and regulate its expression via its GAC 5′UTR. METTL3 increase reduced its translation indicating that increasing levels of METTL3 autoregulates its translation via competing for the 5′UTR GAC motif, which gets outcompeted in doxorubicin cells, either due to increase of competing factors or due to METTL3 protein modification. Overexpression of the m6A demethylase, ALKBH5, decreased translation of all reporters but increased endogenous METTL3 while overexpression of METTL14 decreased general translation. These data showed that METTL3 regulates its own levels via m6A modification of its 5′UTR to promote translation in doxorubicin treated cells, which is reduced upon increased METTL3 levels.

### METTL3 Targets Proliferation and Anti-Viral Response Genes

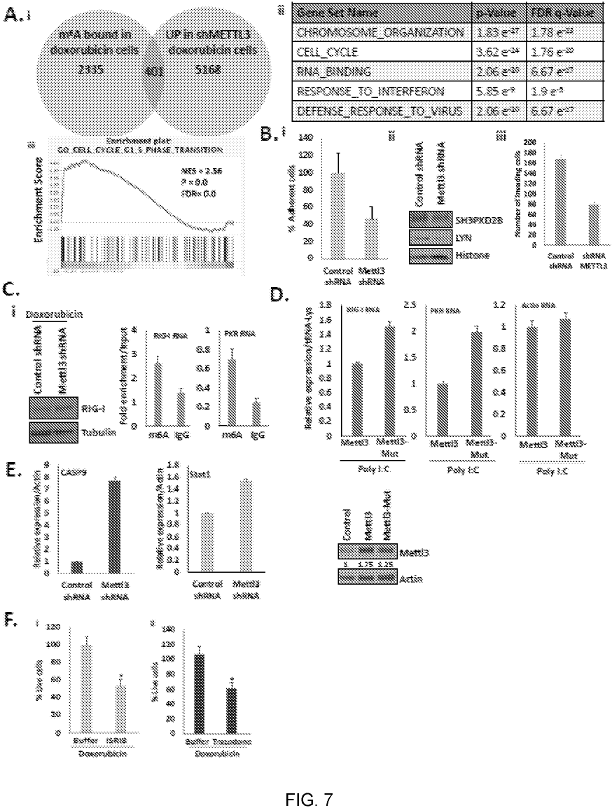

To identify METTL3 targets in G0 and chemoresistance, we conducted global profiling analysis at multiple levels. We identified m6A modified RNA co-immunoprecipitation with antibody against m6A (meRIP) from doxorubicin treated compared to untreated cells, to identify associated target RNAs that bear m6A marks. We also profiled stably transduced shMETTL3 cells compared to control shRNA vector transduced cells upon doxorubicin treatment at the transcriptome and proteome levels to identify genes that are regulated upon METTL3 depletion in chemoresistant cells and selected those that were also m6A targets (1.5 fold and greater) from the meRIP in doxorubicin treated cells but not in untreated cells (FIG. 7A). We found that predominantly, cell cycle genes are significantly decreased by METTL3 at the RNA levels, and are the top category associated with m6A antibody (FIG. 7A); consistently, these increase upon METTL3 depletion in MCF7 cells (FIG. 7A).

As depletion of METTL3 increases cell cycle mRNAs (FIG. 7A), it would render the cells vulnerable to chemotherapy due to increased cell cycle and replication; consistently, we found that METTL3 depletion reduced chemoresistance (FIG. 1D). In contrast, METTL3 overexpression (FIG. 1D) or overexpression of HuR that increase METTL3 (FIG. 5E), promoted chemosurvival, while expression of a catalytically defective mutant of METTL3 (CD-M3) reduced chemoresistance (FIG. 1D). These data are consistent with a role for METTL3 mediated modification of target RNAs that control proliferation to downregulate them; as these proliferation promoting mRNAs are detrimental in the presence of chemotherapy, the increase of METTL3 in chemoresistant cells would downregulate them enabling chemosurvival.

### METTL3 Targets Anti-Viral Response Genes

A second class of genes affected by METTL3 depletion and m6A antibody are anti-viral immune response genes (FIG. 7A) that can trigger cell death. We found that DDX58/RIG-I and PKR that recognize unmodified RNA are decreased in doxorubicin treated cells; consistently, DDX58 and PKR are increased upon METTL3 depletion (FIG. 7B). As m6A methylation on RNA causes RNA to be recognized as self and reduces the anti-viral response, this would complement the loss of m6A in shMETTL3 cells to promote the RIG-I response. These data showed that METTL3 also targets and decreases anti-viral immune response genes in doxorubicin resistant cells.

Such genes may need to be reduced in chemoresistant cells to curb cell death, so that the cells enter G0 and survive anti-proliferation therapy. If METTL3 depletion increases the antiviral response, then overexpression of METTL3 would reduce anti-viral response. This would also be consistent and complementary to the role of m6A in increased antiviral response in its absence. We tested this by overexpressing either METTL3 or the catalytically defective mutant CD-M3, followed by poly I:C treatment to test for anti-viral response gene upregulation by qPCR. We found that compared to the catalytically defective mutant CD-M3 expressing cells, cells overexpressing METTL3 reduced the antiviral response genes of RIG-I and PKR (FIG. 7C). These data showed that stress signals increase METTL3 to suppress the anti-viral response, where the increased METTL3 downregulates these genes. The RIG-I pathway can trigger anti-viral immune response and can contribute to cell death of METTL3 depleted cells. Consistently, we found that CASP9 and STAT1 downstream targets of RIG-I that mediate cell death and anti-viral immune response are increased in METTL3 depleted cells (FIG. 7E); this is in accord with increased cell death on RIG-I increase due to loss of m6A regulation in METTL3 depleted cells. These data are consistent with the role of m6A in reducing cell death to promote survival.

### METTL3 Promotes Cell Adhesion Genes

m6A can also promote non-canonical translation. Therefore, we examined our meRIP datasets compared to Mettl3 depletion RNA profiles and proteomic datasets for m6A target genes that are not disrupted at the RNA level but are decreased upon METTL3 depletion. These would be m6A targets that are promoted at the protein or translation level in the presence of METTL3 in doxorubicin treated cells. We found that cell adhesion and invasion genes that are associated with metastasis are m6A targets that are downregulated upon METTL3 depletion at the protein level but not RNA level (FIG. 4E). Therefore, we tested whether METTL3 was required for cell invasion and adherence of doxorubicin surviving cells, given that it is established that doxorubicin resistant cells show increased invasiveness. Consistently, we found that METTL3 depletion decreases adherence and invasiveness of these cells (FIG. 7D), consistent with decrease of such genes (FIG. 7D). These data together showed that METTL3 increase, as a stress response via ISR in doxorubicin surviving cells, can promote cell invasion and tumorigenesis via upregulation of such genes at the protein level.

### Overriding EIF2α Phosphorylation Reduces Chemosurvival

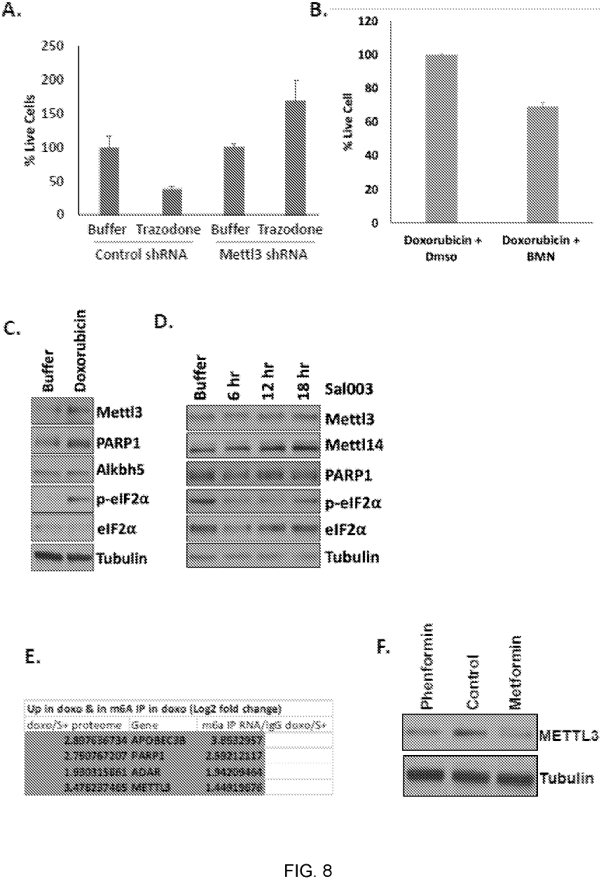

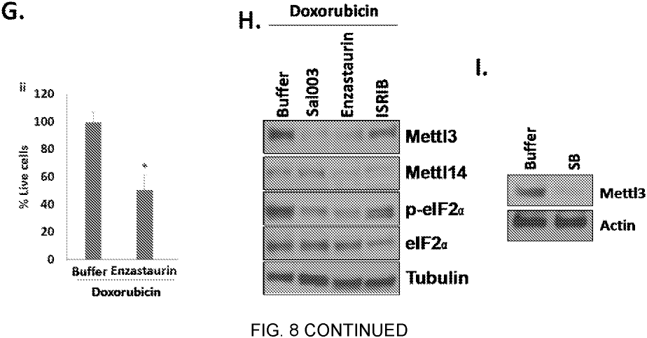

Our data showed that stress signals in chemotherapy treated cells cause elF2α phosphorylation that enables METTL3 upregulation, which alters gene expression to promote resistance by suppressing cell cycle genes. Therefore, pharmacological inhibitors that override elF2α phosphorylation (ISRIB, trazodone), could alleviate chemoresistance. Consistent with our results with METTL3 depleted cells, these cells treated with trazodone or ISRIB that bypass the effect of elF2α phosphorylation and prevent the increase of METTL3 in FIGS. 3D and 3E, correspondingly show significant decrease in chemosurvival (FIG. 4F). This is also observed with Metformin that has been shown to be an inhibitor of PKR that reduces elF2α phosphorylation. Consistently, we see decreased induction of METTL3 with metformin and phenformin addition to doxorubicin treated cells and decreased chemosurvival (FIG. 8F). Synergistic decrease in chemosurvival was not observed upon METTL3 depletion and trazodone, indicating that the elF2α pathway mediates resistance at least in part through METTL3 (FIG. 8A). This is consistent with the increase of antiviral and cell cycle genes upregulated in shMETTL3 cells that correlate with decreased chemosurvival. These data showed that chemoresistance can be reduced by suppressing the effect of the elF2α phosphorylation pathway. This reduces METTL3 and thus prevents m6A regulation of mRNAs that need to be controlled to support chemosurvival.

### PARP1 DNA Repair Gene and RNA-DNA Editors, ADAR and APOBEC3B, are Increased In Doxorubicin Treated Cells and in m6A Immunoprecipitates and Affect Tumor Chemo- and Immune-Survival

We compared the proteome dataset of increased in doxorubicin treated cells compared to untreated with our m6A immunoprecipitates, to identify those genes that are upregulated in doxorubicin treatment and are promoted by m6A. While this revealed the cell adhesion genes mentioned above, it also yielded the DNA repair genes such as PARP1, and editing enzymes, ADAR and APOBEC3B (FIG. 8E). Consistently, doxorubicin treated cells are sensitive to PARP inhibitors, such as Talazoparib (BMN-673, FIG. 8B). We found that PARP1 increases in doxorubicin treated cells and in elF2α phosphatase inhibitor, Sal003 treated cells (FIGS. 8C and 8D), consistent with these data that showed that METTL3 increase in doxorubicin cells promotes PARP1 increase and PARP inhibitor sensitivity.

APOBEC3B increase is known to cause escape from therapy due to mutational plasticity via the mutations induced by this DNA editor that causes cytosine to uridine changes in the DNA leading to thymidine mutations that can evade multiple types of therapy. However, this would also render such doxorubicin cells that enhance APOBEC3B and C-T mutated neoeipitopes and is susceptible to immunotherapy in the context of immune checkpoint blockade, as well as to ATR inhibitor and other DDR inhibitors. This would render cells also sensitive to PARP inhibitors and other DDR inhibitors, consistent with our data in FIG. 8B.

We found ADAR1, an RNA Cytosine to Uridine editor, enhanced. This would modify RNAs and render RNAs less susceptible to triggering anti-viral receptors that are also consistently decreased by METTL3 (FIGS. 7B and 7C), leading to reduced anti-viral immune and apoptotic responses. Consistently, using 8-azaadenosine, an inhibitor of ADAR, or phenformin that reduces elF2α phosphorylation (FIG. 8F), reduced doxorubicin survival.

APOBEC3B would lead to neoepitopes that could trigger the immune system. However, this is not readily seen as inhibitors of immune cells such as TGF-β that inhibits NK cells are increased in doxorubicin treated breast cancer cells. Additionally, as seen in our m6A immunoprecipitates, m6A downregulates a number NK activating receptors and T cell immunomodulators, which would disable anti-tumor immunity. M6A also downregulates antiviral innate immune response while promoting ADAR1 that suppresses antiviral immunity. Therefore, inhibition of METTL3 would activate them. Therefore, we co-cultured untreated or doxorubicin or gemcitabine treated cells that were also treated with trazodone or metformin or ISRIB or phenformin (to reduce elF2α phosphorylation and block METTL3 effects), or METTL3 depleted cells with CD14+ monocytes, CD8+ T cells, and NK (NK92) cells to observe loss of survival. Blocking ADAR1 by using 8-azaadenosine also renders the cells sensitive to anti-tumor immunity, when co-cultured with CD14+ monocytes, indicating that METTL3 promotes ADAR for tumor survival. These data indicate that METTL3 and m6A override chemosensitivity and anti-tumor acquired immunity.

### Overriding PKC Reduces Chemosurvival

HuR is modified by a number of stress signaling pathways, including DNA damage downstream kinases, PKC and p38 MAPK that are activated by stress conditions such as chemotherapy and can promote its cytoplasmic localization. HuR is also phosphorylated by PKC under stress conditions such as doxorubicin chemotherapy that activate PKC, which is needed for cytoplasmic localization. As doxorubicin treated cells show increased phosphorylation of PKC, we found that such cells also treated with Enzastaurin, a PKC inhibitor, show reduced METTL3 levels and survival (FIGS. 8G and 8H), compared to buffer treated cells. Together, these data indicate that HuR is needed for translation of METTL3 levels in doxorubicin resistant cells.

### SUMMARY

We have found that post-transcriptional RNA stability changes due to increase of specific RNA regulators. Our data revealed that the RNA m6A methyltransferase, METTL3, and co-factor, METTL14, increase transiently in cancer cell lines and patient samples treated with chemotherapy to isolate chemosurviving cells (FIGS. 1A, 1E, 1F, and 2A-2D). This was observed with doxorubicin treatment of triple negative and hormone positive breast cancer cell lines and patient samples as well as with cytarabine in leukemic THP1 cells. This is also replicated in conditions that inhibit mTOR/Pl3K (FIGS. 4BE) where elF2α is also phosphorylated. Consistently, activators of the integrated stress response pathway, such as poly I:C that causes elF2α phosphorylation, promotes METTL3 and METTL14 levels (FIGS. 2A, D, S2A). Consistent with increased METTL3, m6A was increased on RNA in chemosurviving cells, in cells with ISR activator poly I:C, or with elF2α phosphatase inhibitor, but not with METTL3 depletion (FIGS. 1B, 3B, 2E). We found that m6A modification on mRNAs leads to post-transcriptional downregulation of genes that need to be suppressed in the presence of chemotherapy to enable chemosurvival. These include cell cycle genes that would lead to cell death in the presence of chemotherapy that targets the cell cycle and antiviral immune response genes that can trigger cell death (FIGS. 7A-7F and 8A-8H). These data showed that chemoresistant cells inhibit canonical translation, and increase METTL3 and METTL14 via non-canonical translation, to regulate gene expression that contributes to chemosurvival.

Therapy induced DNA damage and stress signaling leads to activation of integrated stress response pathway via elF2α kinases, which we found increases METTL3 and METTL14 (FIGS. 1A-1F, 2A-2G, 3A-3J, and 4A-4H). Consistently, the increased METTL3 and METTL14 in chemotherapy treated cells such as doxorubicin treated cells, can be mimicked by mTOR inhibitors Torin1 and Bez 235 that are known to lead to phosphorylation of elF2α, as well as by integrated stress response inducers such as poly I:C. The effects of doxorubicin or elF2α phosphorylation are transient stress responses (FIG. 2A) and METTL3 levels are subsequently restored. These stress signals phosphorylate elF2α; consistently, a phosphatase inhibitor, Sal003 that blocks dephosphorylation of elF2α, also increases METTL3 and METTL14 (FIG. 3C). Furthermore, inhibitors that override the effects of elF2α phosphorylation, prevent METTL3 and METTL14 increase (FIGS. 3D and 3E). These include Trazodone that affects the ternary complex or ISRIB that activates elF2B to be insensitive to the effects of elF2α phosphorylation.

With mTOR activity inhibited and elF2α phosphorylated, canonical translation is reduced. These changes allow non-canonical expression of mRNAs which are recruited by specific RNA binding protein complexes. We identified RNA binding proteins, HuR and translation factor, elF3a, as associated with METTL3 mRNA. These proteins were also associated with METTL14 mRNA that is co-regulated in expression with METTL3. HuR is an RNA binding protein that is increased upon genotoxic stress such as doxorubicin treatment and can shuttle out of the nucleus leading to RNA stability and translation increase. Consistently, we found that HuR-S221D, a cytoplasmic form of HuR when overexpressed promotes METTL3 5′UTR reporter translation, endogenous METTL3 levels, and consistently, chemosurvival. HuR can promote translation via direct or indirect association with the 5′UTR or 3′UTR, enabling non-canonical specific mRNA translation under stress conditions. This was also observed with elF3a overexpression. Both proteins are known to bind GAC motifs that are m6A methylated and can promote expression of such mRNAs. This is consistent with our findings that the 5′UTR GAC is at least needed for translation and m6A demethylation reduces METTL3 reporter translation. elF3 can recruit the preinitiation ribosome complex and start translation. Additionally, we found that HuR and elF3 associate with elF2D, an alternate tRNA recruiter that would be able to function under these conditions of compromised elF2α. Consistently, depletion of elF2D or of elF3a or of HuR reduced METTL3 levels and chemosurvival. This showed that elF2α needs to be phosphorylated to reduce its function and canonical translation that is dominant, to enable such non-canonical translation mechanisms mediated via elF2D association with translation factor, elF3a, and RNA binding protein, HuR, on METTL3 mRNA. Consistently, treatment with Sal-003 that phosphorylates elF2α promotes such translation (FIG. 3C) while treatment with ISRIB that overrides the elF2α phosphorylation block to canonical translation, reduces the increase in METTL3 (FIG. 3E).

We further identified that the 5′UTR of METTL3 was needed for doxorubicin and ISR induced non-canonical translation mediated by HuR and elF3A. HuR and elF3A have been demonstrated to bind and promote m6A modified RNA expression, and METTL3 is known to have such 5′UTR GAC sites that respond to non-canonical translation. Therefore, we tested METTL3 5′UTR reporters and identified the 5′UTR was sufficient to confer doxorubicin and ISR responsive translation but not when a GAC motif upstream of the ATG was mutated. Consistently, we found that HuR and elF3A overexpression promoted translation of the 5′UTR reporter but not of the mutated GAC reporter. Confirming that the m6A site was required, we found that overexpression of ALKBH5 that demethylates m6A sites, decreased this 5′UTR reporter translation. The features on METTL14 mRNA that promote translation remain to be explored but the 5′UTR also harbors such GAC sites as does the 3′UTR downstream of the stop codon and HuR and elF3A associate with METTL14 mRNA. Together, these data showed that METTL3 mRNA promotes non-canonical translation via its m6A modified 5′UTR that binds HuR and elF3A.

We found that the targets of m6A (a mediator of post-transcriptional regulation) in doxorubicin treated cells are predominantly cell cycle genes that are decreased at the RNA level (FIG. 7A). This is consistent with enabling chemoresistance, where the cell cycle must be inhibited to avoid cell death due to chemotherapy that targets proliferation. Consistently, we found that METTL3 depletion decreases chemosurvival (FIG. 7B). In support, chemoresistance decreases upon METTL3 depletion with concomitant increase in cell cycle genes (FIG. 1D). Thus, increased METTL3 and METTL14 enables such cells to survive adverse conditions of stress response triggered by chemotherapy by downregulation of proliferation.

Apart from cell cycle genes, our data showed that METTL3 depletion upregulates expression of genes involved in anti-viral immune response, including PKR and the viral RNA/pattern recognition receptor that mediates type-1 interferon response, RIG-I/DDX58 when unmodified viral non-self RNA is present. m6A and other modifications are known to mark RNAs as self RNAs to prevent triggering the cellular anti-viral response. The anti-viral stress immune response can lead to tumor cell death. Consistently, METTL3 depleted cells increase antiviral response and downstream STAT1 and TBK1 signaling (FIG. 7E); conversely, overexpressing METTL3 and challenging with poly I:C to trigger the anti-viral response compared to a catalytic mutant, shows decreased expression of PKR and RIG-I (FIG. 7D). The increase in METTL3 and METTL14 and thus m6A on RNAs, is the mechanism to prevent this induced cell death, both by methylating endogenous RNAs that then do not trigger an anti-viral response as they are recognized as self-RNAs, as well as by downregulating DDX58 and PKR expression. Additionally, METTL3 depletion decreased the expression of a small set of cell adhesion genes at the protein level that would be needed for increased aggressiveness of the chemoresistant tumor; consistently, METTL3 depleted cells show reduced cell adherence (FIG. 7B). Thus, these data showed that METTL3 increases in chemosurviving cells to regulate genes that favor progression and survival of the resistant tumor.

Together, our data showed that chemotherapy induces stress signals (ISR) that trigger shutdown of conventional post-transcriptional mechanisms and enables non-canonical mechanisms of specific genes. This involves RNA binding proteins that are themselves induced by such stress signals (HuR) as well as alternate translation factors (elF2D) that are operational when canonical translation factors are inhibited by stress signals. This induces METTL3 to turn on yet another cascade of events that lead to precise and dynamic change from cell cycling to shutdown of that, of antiviral response that leads to apoptosis, and upregulation of invasion genes. These data showed that chemotherapy stress signals activate the integrated stress response to promote METTL3 and METTL14 via non-canonical translation mediated by elF2α phosphorylation. METTL3 and METTL14 in turn reduces proliferation genes. This allows chemoresistant cells to reduce proliferation to protect them from therapy that would lead to cell death otherwise. This showed METTL3 and METTL14 non-canonical translation as a potential vulnerability of such chemoresistant cells. Reducing METTL3 and METTL14 non-canonical translation, via inhibition of elF2α phosphorylation improves the efficacy of chemotherapy and prevent chemosurvival. Consistently, we found that inhibition of elF2α phosphorylation pathway prevents METTL3 and METTL14 increase (FIGS. 3D and 3E). These inhibitors include trazodone or ISRIB which override the effect of phosphorylated elF2α downstream, or enzastaurin that inhibits PKC that causes elF2α phosphorylation via PKR. HuR is also phosphorylated by PKC, which is needed for its cytoplasmic role; consistently, as show in FIG. 8H we found that doxorubicin treated cells that are also treated with Enzastaurin, a PKC inhibitor, shows reduced METTL3 levels, compared to buffer treated cells. Consistent with decreased METTL3, we found that such cells that are treated with a combination of chemotherapy and trazodone, ISRIB or enzastaurin, reduce chemosurvival significantly (FIG. 8H). Depletion of METTL3 reduces chemosurvival but addition of trazodone did not reduce chemosurvival further, indicating that absence of METTL3 and trazodone are in the same pathway that causes reduced chemoresistance (FIG. 8A). These data indicated potential inhibitors that can be combined to improve the efficacy of chemotherapy by reducing METTL3 mRNA non-canonical translation in these conditions. Together, our data showed that a targetable vulnerability of chemoresistant cells where non-canonical, specialized translation regulates post-transcriptional modification enzymes that control chemoresistance.

### OTHER EMBODIMENTS

In other embodiments, a TGFβ inhibitor may be used to reduce METTL3 levels. TGFβ signaling induces PKR to cause elF2α phosphorylation.

In other embodiments, RNA binding proteins, such as HEXIM1, recruit mRNAs, such as METTL3, that are expressed via non-canonical translation. With mTOR activity inhibited and elF2α phosphorylated, canonical translation that directs proliferation gene expression is reduced. These changes allow non-canonical expression of mRNAs which are recruited by specific RNA binding protein complexes. HEXIM1 increased in these stress conditions, as associated with and required for METTL3 translation. HEXIM1 also associates with tRNAs and is bound to ribosomes as shown by Y10B immunoprecipitation. While HEXIM1 is best known as a repressor of pTEFb, it has also been shown to promote RNA stability and specific mRNA expression in conditions of stress like nucleotide deprivation. HEXIM1 associates with complexes that contain DNA-PK as well as with PPM1G that can respond to ATM activation on DNA damage stress upon chemotherapy or to Akt inhibition when it binds and dephosphorylates 4EBP to prevent cap dependent translation. Consistently, we also found PPM1G associated with METTL3 mRNA in doxorubicin treated but not in untreated cells. PPM1G-HEXIM1 complex may thus be recruited to METTL3 mRNA where the conventional cap dependent complex is inactivated by the PPM1G-4EBP interaction at the cap while HEXIM1 may bring in the translation machinery through its interactions with such factors like tRNAs, elF2β and elF5B. Since the interaction with tRNA and METTL3/14 mRNAs with HEXIM1-PPM1G are best seen in chemotreated but not untreated cells, this suggests that elF2α may need to be phosphorylated to reduce its function and canonical translation that is dominant to enable such non-canonical translation mechanisms mediated via HEXIM1-PPM1G on specific mRNAs. Consistently, treatment with Sal003 that phosphorylates elF2α promotes such translation while treatment with trazodone that overrides the elF2α phosphorylation block to canonical translation, reduces the increase in METTL3 and HEXIM1 association with METTL3 mRNA.

In other embodiments, endogenous retroviral transposons (HERVs) can be induced in stress conditions, such as chemotherapy. HERVs are degraded by RIG-I, as such RNAs can trigger antiviral interferon response. Consistently, our data show that HERVs like ERV3-1 and ERVK13-1 increase in doxorubicin treated cells upon METTL3 increase and m6A self-marking but decrease upon METTL3 depletion and RIG-I increase. The anti-viral stress response can mount an immune response, which can lead to tumor cell death. The increase in METTL3 and METTL14 and thus m6A on RNAs, can prevent this stress-induced cell death by, for example, methylating endogenous RNAs that include HERVs that then do not trigger an anti-viral response as they are recognized as self-RNAs.

In other embodiments, potential inhibitors that can be combined to improve the efficacy of chemotherapy by reducing METTL3 mRNA non-canonical translation include elF2α phosphorylation inhibitors, such as the compounds disclosed in WO 2014/144952 and WO 2014/161808, and METTL3 inhibitors, such as miR600 and other small interfering RNAs based on the METTL3 sequence as disclosed in Chinese Patent CN107349217. With respect to HEXIM1, see, for example, Lew et al., Cancers. 5(3):838-56 (2013) and Shao et al., Mol. Biol. Cell. 31(17):1867-78 (2020).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Other embodiments are within the following claims.

1. A combination comprising:
(i) trazodone and
(ii) a chemotherapeutic.

2. A combination comprising:
(i) an integrated stress response (ISR) overrider, an ISR inhibitor, an adenosine deaminases acting on ribonucleic acid (ADAR) inhibitor, a protein kinase C (PKC) inhibitor, a poly adenosine diphosphate-ribose polymerase (PARP) inhibitor, a methyltransferase-like 3 (METTL3) inhibitor, or a one-carbon metabolism inhibitor; and
(ii) a chemotherapeutic.

3. The combination of claim 1 or 2, wherein the chemotherapeutic is paclitaxel, gemcitabine, cytarabine, doxorubicin, or etoposide.

4. The combination of claim 2 or 3, wherein the combination comprises an ISR overrider.

5. The combination of claim 4, wherein the ISR overrider comprises trazodone or integrated stress response inhibitor (ISRIB).

6. The combination of claim 2 or 3, wherein the combination comprises an ISR inhibitor.

7. The combination of claim 6, wherein the ISR inhibitor comprises metformin or phenformin.

8. The combination of claim 2 or 3, wherein the combination comprises an ADAR inhibitor.

9. The combination of claim 8, wherein the ADAR inhibitor comprises 8-azaadenosine.

10. The combination of claim 2 or 3, wherein the combination comprises a PKC inhibitor.

11. The combination of claim 10, wherein the PKC inhibitor comprises enzastaurin.

12. The combination of claim 2 or 3, wherein the combination comprises a PARP inhibitor.

13. The combination of claim 12, wherein the PARP inhibitor comprises talazoparib.

14. The combination of claim 2 or 3, wherein the combination comprises a METTL3 inhibitor.

15. The combination of claim 14, wherein the METTL3 inhibitor comprises an interfering RNA molecule.

16. The combination of claim 15, wherein the interfering RNA molecule is a short hairpin RNA (shRNA).

17. The combination of claim 15, wherein the interfering RNA molecule is a small interfering RNA (siRNA).

18. The combination of claim 17, wherein the siRNA comprises a target sequence having the nucleic acid sequence of SEQ ID NO: 1.

19. The combination of claim 17 or 18, wherein the siRNA comprises a sense strand having the nucleic acid sequence of SEQ ID NO: 2.

20. The combination of any one of claims 17-19, wherein the siRNA comprises an antisense strand having the nucleic acid sequence of SEQ ID NO: 3.

21. The combination of claim 14, wherein the METTL3 inhibitor comprises rocaglates.

22. The combination of claim 2 or 3, wherein the combination comprises a one-carbon metabolism inhibitor.

23. The combination of claim 22, wherein the one-carbon metabolism inhibitor comprises methotrexate, serine hydroxymethyltranferase inhibitor 1 (SHIN-1), bisantrene, or brequinar.

24. The combination of any one of claims 1 to 23, further comprising immune cells.

25. The combination of claim 24, wherein the immune cells are monocytes (e.g., CD14+ monocytes), T cells (e.g., CD8+ T cells), or Natural Killer cells (e.g., NK92 cells).

26. A method of treating cancer in a subject, the method comprising administering to the subject:
(i) trazodone and
(ii) a chemotherapeutic.

27. A method of treating cancer in a subject, the method comprising administering to the subject:
(i) an ISR overrider, an ISR inhibitor, an ADAR inhibitor, a PKC inhibitor, a PARP inhibitor, a METTL3 inhibitor, or a one-carbon metabolism inhibitor; and
(ii) a chemotherapeutic.

28. The method of claim 26 or 27, wherein the cancer comprises acute myeloid leukemia, liver cancer (e.g., hepatocellular carcinoma or hepatoblastoma), gastric cancer, lung cancer (e.g., non-small cell lung cancer), colorectal cancer, bladder cancer, pancreatic cancer, glioblastoma, prostate cancer, or breast cancer (e.g., triple negative breast cancer or hormone-positive breast cancer).

29. The method of claim 28, wherein the cancer is acute myeloid leukemia.

30. The method of claim 28, wherein the cancer is breast cancer (e.g., triple negative breast cancer or hormone-positive breast cancer).

31. The method of any one of claims 26 to 30, wherein the chemotherapeutic is paclitaxel, gemcitabine, cytarabine, doxorubicin, or etoposide.

32. The method of any one of claims 27 to 31, wherein the method comprises administering an ISR overrider.

33. The method of claim 32, wherein the ISR overrider comprises trazodone or ISRIB.

34. The method of any one of claims 27 to 31, wherein the method comprises administering an ISR inhibitor.

35. The method of claim 34, wherein the ISR inhibitor comprises metformin or phenformin.

36. The method of any one of claims 27 to 31, wherein the method comprises administering an ADAR inhibitor.

37. The method of claim 36, wherein the ADAR inhibitor comprises 8-azaadenosine.

38. The method of any one of claims 27 to 31, wherein the method comprises administering a PKC inhibitor.

39. The method of claim 38, wherein the PKC inhibitor comprises enzastaurin.

40. The method of any one of claims 27 to 31, wherein the method comprises administering a PARP inhibitor.

41. The method of claim 40, wherein the PARP inhibitor comprises talazoparib.

42. The method of any one of claims 27 to 31, wherein the method comprises administering a METTL3 inhibitor.

43. The method of claim 42, wherein the METTL3 inhibitor comprises an interfering RNA molecule.

44. The method of claim 43, wherein the interfering RNA molecule is a shRNA.

45. The method of claim 43, wherein the interfering RNA molecule is a siRNA.

46. The method of claim 45, wherein the siRNA comprises a target sequence having the nucleic acid sequence of SEQ ID NO: 1.

47. The method of claim 45 or 46, wherein the siRNA comprises a sense strand having the nucleic acid sequence of SEQ ID NO: 2.

48. The method of any one of claims 45-47, wherein the siRNA comprises an antisense strand having the nucleic acid sequence of SEQ ID NO: 3.

49. The method of claim 42, wherein the METTL3 inhibitor comprises rocaglates.

50. The method of any one of claims 27 to 31, wherein the method comprises administering a one-carbon metabolism inhibitor.

51. The method of claim 50, wherein the one-carbon metabolism inhibitor comprises methotrexate, SHIN-1, bisantrene, or brequinar.

52. The method of claim 26, wherein trazodone and the chemotherapeutic are co-administered.

53. The method of claim 26, wherein trazodone is administered prior to the chemotherapeutic.

54. The method of any one of claims 27 to 51, wherein the ISR inhibitor, ADAR inhibitor, PKC inhibitor, PARP inhibitor, METTL3 inhibitor, or one-carbon metabolism inhibitor and the chemotherapeutic are co-administered.

55. The method of any one of claims 27 to 51, wherein the ISR inhibitor, ADAR inhibitor, PKC inhibitor, PARP inhibitor, METTL3 inhibitor, or one-carbon metabolism inhibitor is administered prior to the chemotherapeutic.

56. The method of any one of claims 26 to 55, further comprising administering immune cells to the subject.

57. The method of claim 56, wherein the immune cells are monocytes (e.g., CD14+ monocytes), T cells (e.g., CD8+ T cells), or Natural Killer cells (e.g., NK92 cells).

58. Use of an inhibitor of METTL3 or an inhibitor of METTL14 or an inhibitor of eIF2α phosphorylation to decrease cancer cell resistance to chemotherapy.

59. A method of decreasing cancer cell resistance to chemotherapy in a patient comprising administering an inhibitor of METTL3 or an inhibitor of METTL14 or an inhibitor of eIF2α phosphorylation to the patient in an amount sufficient to reduce the resistance of the cancer cell to chemotherapy.

60. A method of treating a cancer in a patient comprising co-administrating (i) a chemotherapeutic agent and (2) a METTL3 or METTL14 inhibitor or a eIF2α phosphorylation inhibitor to the patient.

61. The method or use according to any one of claims 58-60 in which the inhibitor is selected from trazodone, ISRIB, enzastaurin, compounds disclosed in PCT Patent Publications WO2014/144952 and WO2014/161808, miR600 and other small interfering RNAs disclosed in or based upon the METTL3 sequence disclosed in Chinese Patent CN107349217, or combinations of these.

62. Any and all compositions, articles of manufacture, methods and uses disclosed and/or described in this specification.


SEQUENCE LISTING (5 sequences)

SEQ ID NO: 1 - DNA, 21 bp, Artificial Sequence
  Synthetic Construct
cgtcagtatcttgggcaagtt

SEQ ID NO: 2 - RNA, 21 nt, Artificial Sequence
  Synthetic Construct
cgucaguaucuugggcaaguu

SEQ ID NO: 3 - RNA, 21 nt, Artificial Sequence
  Synthetic Construct
aacuugcccaagauacugacg

SEQ ID NO: 4 - DNA, 21 bp, Artificial Sequence
  Synthetic Construct
gctgcacttcagacgaattat

SEQ ID NO: 5 - RNA, 5 nt, Artificial Sequence
  Synthetic Construct
rrach